(19) United States
(12) Patent Application Publication
(10) Pub. No.: US 2026/0048046 A1
(43) Pub. Date: Feb. 19, 2026

(54) METHOD FOR IMPROVING GROWTH PERFORMANCE IN FEEDLOT CATTLE

(71) Applicant: Elanco US Inc., Greenfield, IN (US)

(72) Inventors: Jerold Scott TEETER, Greenfield, IN (US); John Charles KUBE, Greenfield, IN (US); Cory T. HERR, Greenfield, IN (US); Gary VOGEL, Greenfield, IN (US); Phil RINCKER, Greenfield, IN (US); Janet B. ALLEN, Greenfield, IN (US); Michael SAVIDES, Greenfield, IN (US)

(21) Appl. No.: 19/103,921

(22) PCT Filed: Aug. 18, 2022
(86) PCT No.: PCT/US2022/040798

§ 371 Date: Feb. 14, 2025

(51) Int. Cl. A61K 31/4436; A61K 9/00; A61P 3/00

(52) U.S. Cl. CPC A61K 31/4436; A61K 9/0053; A61P 3/00

(57) ABSTRACT

Disclosed a method of improving growth performance in feedlot cattle by administering an effective amount of lubabegron or a pharmaceutically acceptable salt thereof, at a dose of between about 3.5 and 22 mg*kg⁻¹ dry matter. In one aspect, the lubabegron is administered over a period of 56 days for up to 16 to 2 days prior to harvest.

### FIELD OF THE INVENTION

The present invention generally relates to a method of improving growth performance in feedlot cattle by administering an effective amount of lubabegron or a pharmaceutically acceptable salt thereof.

### BACKGROUND OF THE INVENTION

The development of technologies that promote environmental stewardship while maintaining or improving the efficiency of food animal production is essential to the sustainability of producing a food supply to meet the demands of a growing population. Lubabegron was approved by the U.S. Food and Drug Administration in 2018 for reducing NH₃ gas emissions per kg⁻¹ final body weight (BW) and hot carcass weight (HCW) when administered during the last 14 to 91 days on feed. ‘There exists a need in the art to improve growth performance of feedlot cattle. Applicant unexpectedly discovered that in addition to reducing NH₃ emissions, certain doses of lubabegron have a positive impact on growth performance of feedlot cattle, which is not significantly attenuated when lubabegron is removed prior to harvest.

### SUMMARY OF THE INVENTION

The present disclosure relates to a method for improving growth performance of feedlot cattle comprising administering to the cattle an effective amount of lubabegron or a physiologically acceptable salt thereof at a dose of between 3.5 and 22 mg*kg⁻¹ dry matter (3.5 mg*kg⁻¹ is equivalent to about 3.2 g/ton of dry matter). In embodiments the lubabegron is administered for a time period of between 56 and 91 days.

In embodiments, the dose is between about 3.5 and 5.5 mg*kg⁻¹.

In embodiments, the dose is between 5.5 and 22 mg*kg⁻¹.

In embodiments, the administration is for the last 56 days of the finishing period.

In embodiments, the lubabegron is removed for a period of from between 2 and 16 days prior to harvest without significantly attenuating the improved growth performance.

In embodiments, the lubabegron is removed for a period of from between 2 and 16 days prior to harvest, and wherein final body weight (BW), hot carcass weight (HCW), average daily gain (ADG), gain to feed ratio (G:F), dry matter intake (DMI), dressing percentage, ribeye area or longissimus dorsi muscle area (LM area), yield grade (YG), and/or yield grade distribution are not significantly attenuating by the removal of lubabegron.

In embodiments the lubabegron is removed for a period of 16 days prior to harvest without significantly attenuating the improved growth performance.

In embodiments, the administration of lubabegron increases final body weight (BW) by at least 11 kg when compared to control cattle not administered lubabegron.

In embodiments, the administration of lubabegron results in an increase of hot carcass weight (HCW) by at least 15 kg when compared to control cattle not administered lubabegron.

In embodiments, the administration of lubabegron results in an increase in average daily gain (ADG) of between about 11% and 16% when compared to control cattle not administered lubabegron.

In embodiments, the administration of lubabegron increases the gain to feed (G:F) ratio by at least 10% when compared to control cattle not administered lubabegron. In embodiments, the administration increases dry matter intake (DMI) by the cattle by about 2% to 3%.

In embodiments, the administration of lubabegron increases the ribeye area when compared to control cattle not administered lubabegron.

In embodiments, the administration of lubabegron increases the dressing percentage when compared to control cattle not administered lubabegron. In a specific embodiment, the dressing percentage is increased by about 0.9 to 1.3 units.

In embodiments, the administration of lubabegron decreases yield grade (YG) when compared to control cattle not administered lubabegron. In a specific embodiment, YG decreases by about 10% to 11% when compared to control cattle not administered lubabegron.

In embodiments, the administration of lubabegron improves yield grade distribution when compared to control cattle not administered lubabegron. In a specific embodiment, a greater percentage of cattle are Yield Grade 2 when compared to control cattle not administered lubabegron. In embodiments, a lower percentage of cattle are Yield Grade 4 when compared to control cattle not administered lubabegron.

In embodiments, the lubabegron is administered in feed.

In embodiments, the lubabegron is administered as lubabegron fumarate.

In embodiments, the disclosure provides a method of reducing kidney pelvic and heart fat (KPH) in feedlot cattle comprising administration of an effective amount of lubabegron or a physiologically suitable salt thereof.

In a embodiments, the dose of lubabegron administered is about 22 mg*kg⁻¹ to reduce KPH.

Other objects and features will be in part apparent and in part pointed out hereinafter.

### DETAILED DESCRIPTION

### Methods of Improving Growth Production

The present disclosure provides a method for improving the growth performance in feedlot cattle by administering an effective amount of lubabegron or a salt thereof.

The methods of assessing improved production are known in the art. A skilled artisan will recognize that improved production may be measured comparing the health, weight, size, meat quality, and other parameters between subjects receiving the immunomodulator composition and those subjects not receiving the immunomodulator composition.

In embodiments, the lubabegron is administered as lubabegron fumarate. However, because lubabegron contain a basic moiety, it can also exist as a pharmaceutical acid addition salt. Such salts include the salicylate, sulfate, pyrosulfate, bisulfate, sulfite, bisulfite, phosphate, mono-hydrogenphosphate, dihydrogenphosphate, metaphosphate, pyrophosphate, chloride, bromide, iodide, acetate, propionate, decanoate, caprylate, acrylate, formate, isobutyrate, heptanoate, propiolate, oxalate, malonate, succinate, suberate, sebacate, maleate, 2-butyne-1,4 dioate, 3-hexyne-2,5-dioate, benzoate, chlorobenzoate, hydroxy benzoate, methoxy benzoate, phthalate, xylenesulfonate, phenylacetate, phenylpropionate, phenylbutyrate, citrate, lactate, hippurate, β-hydroxy butyrate, glycolate, maleate, tartrate, methanesulfonate, propanesulfonate, naphthalene-1-sulfonate, naphthalene-2-sulfonate, mandelate and like salts. Preferred acid addition salts include the hemi-fumarate, benzoate, salicylate, R-mandelate, hydrochloride and glycolate salts.

### Definitions

Lubabegron, has the chemical name of 2-{4-[2-({(2S)-2-Hydroxy-3-[2-(2-thienyl)phenoxy]propyl}amino)-2-methylpropyl]phenoxy}nicotinonitrile. Lubabegron free base is assigned CAS No.: 391920-32-4.

The term “effective amount” refers to the amount necessary or sufficient to realize a desired biologic effect. For example, an effective amount of lubabegron will be the amount required to elicit the improvements growth performance of the feedlot cattle mentioned below.

The term “elicit” can be used interchangeably with the terms activate, stimulate, generate or upregulate.

The term “body weight” may refer to unshrunk body weight or shrunk body weight. Shrink is imposed on cattle to eliminate gastrointestinal fill.

The term “hot carcass weight” is the weight of the unchilled carcass in pounds after the head, hide and internal organs have been removed.

The term “finishing period” or “feeding period” is the period of time when cattle are feed a high energy diet to promote weight gain and muscle production and optimize fat cover prior to slaughter.

The term “average daily gain” or “ADG” is the average amount of weight an animal will gain each day during the feeding or finishing period. It can be calculated by taking the amount of weight the animal gained since the previous weight measurement and dividing the weight by the number of days since the last weight. In embodiments, the CPE mean for unshrunk initial and final BW is used to calculate ADG.

The “dressing percentage” is the percentage of the live animal weight that becomes the carcass weight at slaughter. It is calculated by dividing the HCW by the shrunk live weight of the animal and expressing the value as a percentage (multiply by 100).

The “gain to feed ratio” or “G:F” is he amount of feed an animal consumes as compared to the amount of body weight gained, expressed as a ratio. For example, G:F is calculated as a quotient of ADG divided by the dry matter intake (DMI).

The “ribeye area” is the surface area of the longissimus dorsi muscle between the 12th and 13th rib of a beef or lamb carcass. This is also referred to as longissimus area or “LA.”

The “marbling score” or “MS” is the visual assessment of visible fat found between muscle fiber bundles within the ribeye muscle.

“Feedlot” cattle as used herein include bulls, steers, cows, heifers and ovariectomized heifers.

“Yield grade” or “YG” is an estimate of the amount of boneless, closely trimmed retail cuts from high-value parts of a carcass, i.e., round, loin, rib, and chuck. Yield grades are rated numerically and are 1, 2, 3, 4, and 5. A Yield Grade 1 carcass is the leanest whereas a Yield Grade 5 carcass is the fattest. The yield grade of a carcass is determined by: 1) external fat: 2) kidney, heart and pelvic fat: 3) ribeye area; and 4) hot carcass weight. Yield grade can be determined by using the following formula: 2.50+(2.5×adjusted fat thickness in inches)+(0.2×percent kidney, heart and pelvic fat)+(0.0038×hot carcass weight)−(0.32× ribeye area in square inches).

“Yield grade distribution” is the percentage of cattle in a particular yield grade, i.e., 1, 2, 3, 4, or 5, relative to the total number cattle graded. An improved yield grade distribution is achieved when an intervention results in one or more cattle in the relevant population having a yield grade number lower than would have been observed absent the intervention.

With respect to improved growth performance due to lubabegron administration, “not significantly attenuated” means no statistically significant change (P<0.05) in improved growth performance, however measured, in feedlot cattle removed from lubabegron between 2 and 16 days prior to harvest as compared to what would be observed and/or expected in feedlot cattle not removed from lubabegron between 2 and 16 days prior to harvest.

When introducing elements of the present invention or the preferred embodiments thereof, the articles “a”, “an,” “the” are intended to mean that there are one or more of the elements. The terms “comprising”, “including” and “having” are intended to be inclusive and mean that there may be additional elements other than the listed elements.

### Administration and Dosage

In embodiments, the lubabegron or a physiologically acceptable salt thereof is administered to the feedlot cattle in feed.

While embodiments for administration of lubabegron, or a physiologically acceptable salt thereof, are via daily feed, lubabegron, or a physiologically acceptable salt thereof, can be incorporated into salt blocks and mineral licks, as well as being added directly to lick tank formulations or drinking water for convenient oral consumption. Lubabegron, or a physiologically acceptable salt thereof, may be administered orally by bolus or gavage.

In embodiments, a feed additive is provided that includes lubabegron, or a physiologically acceptable salt thereof, and one or more suitable carriers. The feed additive may be a dry feed additive or a liquid feed additive. The feed additive, when added with other minerals, forms an animal feed that results in the desired concentration of lubabegron or a physiologically acceptable salt thereof in the animal feed and/or Ingestion of the animal feed is formulated to form an animal feed that yields the desired dose of lubabegron or a physiologically acceptable salt thereof for cattle. For certain feed additives, premixes are a widely accepted term in the art. The premix may be solid or liquid. Mineral premixes are compositions for animal feed formulations and contain the desired type and amount of minerals, especially trace elements. The vitamin premix is a composition for animal feed formulations and contains the desired type and amount of vitamins. Some premixes contain both vitamins and minerals. As such, feed additives contain premixes such as, for example, mineral premixes, vitamin premixes, and premixes containing both vitamins and minerals.

In embodiments lubabegron, or a physiologically acceptable salt thereof, is orally administered to cattle for a suitable period of time, for example 1 or more days. In embodiments, the dosing period may be any number of days between 1 and 100 days. For example, the dosing period may be at least 1 day, at least 10 days, at least 20 days, at least 30 days, at least 40 days, at least 50 days, at least 60 days, at least 70 days, at least 80 days, at least 90 days, or at least 100 days. In embodiments, lubabegron, or physiologically acceptable salt thereof, is administered for at least 91 days. In some embodiments, the lubabegron or a physiologically acceptable salt thereof, is administered for at least 56 days. In embodiments, the lubabegron or physiologically acceptable salt thereof is administered for any period of time between 56 and 91 days, including 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days.

In embodiments, the dosing period ends with a bovine slaughter. The dosing period may, for example, fall within the last 10, 20, 30, 40, 50, 60, 70, 80, 90, or 100 days immediately prior to slaughter. In embodiments, the dosing period may be the 56- or 91-day periods occurring immediately prior to slaughter. In embodiments, the lubabegron or physiologically acceptable salt thereof is administered for any period between the last 56 and 91 days prior to slaughter, including the last 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In other embodiments, orally administered lubabegron, or a physiologically acceptable salt thereof, is discontinued, or removed, one or more days prior to slaughter. For example, lubabegron may be removed between 2 and 16 days prior to slaughter.

In embodiments, the lubabegron, or physiologically acceptable salt thereof, is administered at a dose that provides between about 1.5 to 22 mg*kg⁻¹ dry matter.

In embodiments, the lubabegron, or physiologically acceptable salt thereof, is administered at a dose that provides between about 3.5 to 5.5 mg*kg⁻¹ dry matter.

The dosing for lubabegron may alternatively be expressed in grams per ton of dry matter. 1 gram per ton of dry matter equals approximately 1.102 mg*kg⁻¹ of dry matter. Accordingly, in embodiments, the lubabegron physiologically acceptable salt thereof is generally administered between about 1.36 to 19.96 grams per ton of feed, between about 3.18 and 19.96 grams per ton of feed, or between about 4.99 and 19.96 grams per ton of feed.

Delivery systems may also include non-polymer systems that are lipids including sterols such as cholesterol, cholesterol esters, and fatty acids or neutral fats such as mono-, di-, and tri-glycerides: hydrogel release systems: silastic systems: peptide based systems: wax coatings: compressed tablets using conventional binders and excipients: partially fused implants; and the like. Specific examples include, but are not limited to, erosional systems in which an agent of the invention is contained in a form within a matrix such as those described in U.S. Pat. Nos. 4,452,775, 4,675,189, and 5,736,152, and diffusional systems in which an active component permeates at a controlled rate from a polymer such as described in U.S. Pat. Nos. 3,854,480, 5,133,974, and 5,407,686. In addition, pump-based hardware delivery systems can be used, some of which are adapted for implantation.

The lubabegron, or pharmaceutically acceptable salt thereof, can be administered with other vitamins, minerals, and medicines as necessary. In embodiments, the lubabegron is administered with monensin and tylosin.

In embodiments, the lubabegron, or physiologically acceptable salt thereof, is administered during the finishing period.

In embodiments, lubabegron, or physiologically acceptable salt thereof, is administered during the last 91 days of the finishing period.

In a embodiments, the lubabegron, or physiologically acceptable salt thereof, is administered during the last 56 days of the finishing period.

In embodiments, the lubabegron or physiologically acceptable salt thereof is administered for any period between the last 56 and 91 days of the finishing period including the last 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days.

In embodiments, the lubabegron is removed from the e.g., feed for a period of time between 2 and 16 days prior to harvest without significantly attenuating the positive growth performance.

In embodiments, the lubabegron is removed from the e.g., feed for a period of time between 2 and 16 days prior to harvest without significantly attenuating the positive growth performance in final body weight (BW), hot carcass weight (HCW), average daily gain (ADG), gain to feed ratio (G:F), dry matter intake (DMI), dressing percentage, ribeye area or longissimus dorsi muscle area (LM area), yield grade (YG), and/or yield grade distribution.

In embodiments, the lubabegron is removed for 2 days, 3 days, 4 days, 5 days, 6 days, 7 days, 8 days, 9 days 10 days, 11 days 12 days, 13 days, 14 days, 15 days, or 16 days prior to harvest.

In embodiments, the lubabegron is removed for 2 days, 4 days, 6 days, 8, days, or 16 days prior to harvest.

In a embodiments, the lubabegron is removed for 16 days prior to harvest.

### Evaluating Growth Performance

Growth performance following administration of lubabegron or a physiologically acceptable salt thereof can be measured using methods and analysis known to those skill in the art, including the methods used in the examples below.

In embodiments, the administration of lubabegron or a physiologically acceptable salt thereof improves or increases in one or more of the following: final body weight (BW), hot carcass weight (HCW), average daily gain (ADG), gain to feed ratio (G:F), dry matter intake (DMI), dressing percentage, ribeye area or longissimus dorsi muscle area (LM area), yield grade (YG), and/or yield grade distribution.

In embodiments, the administration of lubabegron or a physiologically acceptable salt thereof reduces kidney pelvic and heart fat (KPH).

### Exemplary Embodiments of the Disclosure

The following paragraphs describe exemplary embodiments of the present disclosure.

1. A method for improving growth performance of feedlot cattle comprising administering to the cattle an effective amount of lubabegron or a physiologically acceptable salt thereof at a dose of between 3.5 and 22 mg*kg⁻¹ dry matter.

2. The method according to embodiment 1, wherein the lubabegron or a physiologically acceptable salt thereof is administered at a dose of 5.5 mg*kg⁻¹ dry matter.

3. The method according to embodiments 1 or 2, wherein the administration is for 56 to 91 days.

4. The method according to embodiment 3, wherein the administration is for the last 56 to 91 days of the finishing period.

5. The method according to any one of embodiments 1-3, wherein the lubabegron is removed for a period of from between 2 and 16 days prior to harvest, and wherein the positive growth performance is not significantly attenuated by the removal of lubabegron.

6. The method according to any one of embodiments 1-3, wherein the lubabegron is removed for a period of from between 2 and 16 days prior to harvest, and wherein final body weight (BW), hot carcass weight (HCW), average daily gain (ADG), gain to feed ratio (G:F), dry matter intake (DMI), dressing percentage, ribeye area or longissimus dorsi muscle area (LM area), yield grade (YG), and/or yield grade distribution are not significantly attenuated by the removal of lubabegron.

7. The method according to embodiments 5 or 6, wherein the lubabegron is removed 16 days prior to harvest.

8. The method according to any one of embodiments 1 to 7, wherein the administration increases final body weight (BW) by at least 11 kg when compared to control cattle not administered lubabegron.

9. The method according to any one of embodiments 1 to 8, wherein the administration results in an increase of hot carcass weight (HCW) by at least 15 kg when compared to control cattle not administered lubabegron.

10. The method according to any one of embodiments 1 to 7, wherein the administration results in an increase in average daily gain (ADG) of between about 11% and 16% when compared to control cattle not administered lubabegron.

11. The method according to any one of embodiments 1 to 7, wherein the administration increases the gain to feed (G:F) ratio by at least 10% when compared to control cattle not administered lubabegron.

12. The method according to any one of embodiments 1 to 7, wherein the administration increases dry matter intake (DMI) by the cattle by about 2% to 3% when compared to control cattle not administered lubabegron.

13. The method according to any one of embodiments 1 to 7, wherein the administration increases the ribeye area when compared to control cattle not administered lubabegron.

14. The method according to any one of embodiments 1 to 7, wherein the administration increases the dressing percentage when compared to control cattle not administered lubabegron.

15. The method according to embodiment 14, wherein the dressing percentage is increased by about 0.9 to 1.3 units.

16. The method according to any one of embodiments 1 to 7, wherein the administration decreases yield grade (YG) when compared to control cattle not administered lubabegron.

17. The method according to embodiment 16, wherein the YG decreases by about 10% to 11% when compared to control cattle not administered lubabegron.

18. The method according to any one of embodiments 1 to 7, wherein the administration improves yield grade distribution when compared to control cattle not administered lubabegron.

19. The method according to embodiment 18, wherein a greater percentage of cattle are Yield Grade 2 when compared to control cattle not administered lubabegron.

20. The method according to embodiment 18, wherein a lower percentage of cattle are Yield Grade 4 when compared to control cattle not administered lubabegron.

21. The method according to any one of embodiments 1 to 20, wherein the wherein the physiologically acceptable salt is lubabegron fumarate.

22. The method according to any one of embodiments 1 to 21, wherein the lubabegron or the physiologically acceptable salt thereof is administered in feed.

23. A method of reducing kidney pelvic and heart fat (KPH) in feedlot cattle comprising administering to the cattle an effective amount of lubabegron or a physiologically acceptable salt thereof.

24. The method according to embodiment 23, wherein the lubabegron or a physiologically acceptable salt thereof is administered in a dose of 22 mg*kg⁻¹.

525. The method according to embodiment 23 or 24, wherein the physiologically acceptable salt is lubabegron fumarate.

As various changes could be made in the disclosed compositions, products, and methods without departing from the scope of the invention, it is intended that all matter contained in the above description and in the examples given below shall be interpreted as illustrative and not in a limiting sense.

The following non-limiting examples are provided to further illustrate the present invention.

### EXAMPLES

### Example 1:91-Day Study of Lubabegron on Growth Performance

### Experimental Design and Treatment

A randomized complete block design was used to evaluate the effect of lubabegron (LUB) on performance over a 91-d period using 336 beef cattle (BW=453±34.5 kg) housed in cattle pen enclosures (CPE). Four LUB treatments were included in the study based on dose: 0.0 (control or CON), 1.38, 5.5, and 22.0 (mg·kg⁻¹ of DM). Because there was a limited number of CPE (n=8), three sequential cycles (blocks) were required to generate 6 replicates of each dose×sex combination. As such, 112 cattle (56 steers and 56 heifers) were housed concurrently within each cycle across the eight CPE (14 cattle/CPE), with each dose×sex combination being represented by a single CPE/cycle. To assure different frame sizes of cattle were represented, cattle in cycles one and three were large-frame Continental crossbreds, whereas cattle in cycle 2 were medium-frame British crossbreds.

Treatment administration for the three cycles occurred from April through July, August through November, and December through March of 2014 and 2015, respectively. Four weeks before beginning treatments for each cycle (day −28), up to 145 cattle were sourced from a common origin and transported to the study site at the University of California-Davis to be group-housed in single-sexed outdoor pens. The presence of growth-promoting implants was assessed, and any preexisting implants were excised before shipment to the study site to ensure they had been implant-free for a minimum of 28 d before treatments began on day 0. No additional implants were used in these cattle, and they were considered nonimplanted.

On day −8, cattle were screened for abnormal health conditions by a veterinarian and ranked by BW to identify the 56 eligible cattle within each sex that provided the narrowest weight range. The following day (day −7), the 56 cattle selected for study enrollment. within each sex were grouped into sets of four consecutively weight-ranked cattle. Cattle were then randomly allocated to their respective treatment within each weight group and transferred into the CPE. The CPE were randomly assigned to sex and dose treatment before each cycle, and all study personnel were blinded to treatments throughout the duration of the study. All cattle were fed the negative control basal finishing diet (Table 1) for 1 week (days −7 to −1) after being placed into the CPE to allow for acclimation before beginning treatments.

Treatments began on day 0 and cattle in each CPE received their respective treatment for 91 d. Emission measurements began at 0800 hours on day 0 and ended at 0500 hours on day 91, immediately preceding cattle removal from the CPE for final BW measurements and transportation to the commercial slaughter facility.

TABLE 1
Ingredient composition (DM basis) and analyzed nutrient content
of the finishing diet fed during the 91-d treatment phase?

| Ingredient | % of DM |
|---|---|
| Ground corn [symbol] | 2.5 |
| Dried distiller's grains with solubles | 10.0 |
| Steam-flaked corn | 63.5 |
| Tallow | 3.0 |
| Cane molasses | 6.0 |
| Alfalfa | 6.0 |
| Wheat straw | 6.0 |
| Limestone | 14 |
| Urea | 1.1 |
| Salt | 0.3 |
| Trace minerals [symbol] | 0.2 |
| Total | 100.0 |
| Analyzed nutrient content, DM basis | |
| DM | 76.5 |
| CP, % of DM | 14.2 |
| Ca, % of DM | 0.66 |
| P % of DM | 0.31 |
| Calculated RDP [symbol] % of DM | 9.34 |
| Calculated RUP [symbol] % of DM | 4.86 |
| Calculated NE [symbol] Mcal [symbol] kg [symbol] DM | 2.21 |
| Calculated NE [symbol] Mcal [symbol] kg [symbol] DM | 1.54 |

[symbol]

Water was included at 7.5% of as-fed feed to reduce likelihood of segregation of ingredients within the type C feed. [symbol]
Ground corn was fed without LUB in the negative control treatment group and served as the carrier for LU [symbol] in the 1.38, 5.5, and 22.0 mg [symbol] kg [symbol] treatment groups. [symbol]
Formulated to contain: 90.60% MgO, 5.05% MnSO [symbol] , 2.31% CuSO [symbol] , 1.98% Zno, 0.03% K [symbol] , 0.02% Na [symbol] SeO [symbol] , and 0.01% CoSO [symbol] . [symbol]
RDP, rumen degradable protein; RUP rumen undegradable protein.
The sum of RDP and RUP is CP. The RDP, RUP, NEm, and NEg were calculated based on the NRC (2000). [symbol]
indicates data missing or illegible when filed

### Cattle Pen Enclosures

The CPE were dome shaped, 22.0×11.3 m structures oriented east to west, standing 6 m tall at the highest point and constructed with a steel frame, welded truss arches with parallel steel tubes, and continuous structural webbing (11 m Legend Series Cover-All Building, Saskatoon, Saskatchewan, Canada), which was covered with a double stacked Dura-Weave cover (Intertape Polymer Group, Montreal, QC, Canada). Each CPE contained 185 m² of soil surface, 9.1 m linear bunk space on a concrete apron, 3% slope from the bunk to the west of the pen, and a float-activated waterer. Two hinged bunk flaps were used to facilitate feed delivery, and each CPE had two doors. There was one large roll-up door to move cattle in and out of the CPE, and one small door to allow study personnel access to the CPE. Both doors and the bunk flaps remained closed when not in use to prevent disruption of CPE gas equilibrium.

The CPE were thoroughly cleaned before each study cycle began by allowing the existing manure to air dry for 24 to 48 h, and then removing the manure with a skid-loader and power washer. The pen floor was leveled and thoroughly saturated with water to allow volatilization of preexisting NH₃ from the soil. Fresh soil was applied following a 24-h volatilization period and then compacted with a weighted roller to create a solid pen surface. Accumulation of excreta began on day −7 when cattle were allocated to the CPE and remained uninterrupted for the entirety of the 91-d gas emission measurement period for each cycle.

### CPE Airflow and Gas Measurements

Each CPE was equipped with a 4.9×1.2 m cooling pad on the east side for evaporative cooling of incoming ambient air, plus two ventilation fans on the west side to create directional airflow and generate negative pressure inside the CPE. Flow rates were independently determined for all 16 ventilation fans before and after each cycle using a customized purpose-built anemometer and the sum of the two fans within a CPE determined total outflow for each respective CPE. Fan efficiency decay curves were created for the determination of airflow at any given time using the two flow rates obtained at the beginning and end of a cycle. Fan speed was monitored continuously using two sensors (Monarch Instruments, Amherst, NH), and the static differential pressure between internal and external air was monitored to ensure proper ventilation. The temperature (T) and relative humidity (RH) within CPE were monitored every 15 s during emission sampling periods (Table 2) using RH/T sensors (Dwyer Instruments, Inc., Michigan City, IN), and the same measurements were obtained continuously from ambient air using an on-site weather station (Novalynx, Model 110-WS-16, Auburn, CA).

TABLE 2
Maximum, minimum, and mean daily ambient temperature (TA), relative
humidity (RH) and temperature humidity index (THI)¹

| Source/ | TA, ° C. | | | RH, % | | | THI² | | |
|---|---|---|---|---|---|---|---|---|---|
| cycle | Max. | Min. | Mean | Max. | Min. | Mean | Max. | Min. | Mean |

Ambient air

| | | | | | | | | | |
|---|---|---|---|---|---|---|---|---|---|
| Cycle 1 | 42.5 | 10.1 | 23.9 | 70.4 | 12.9 | 47.7 | 86.2 | 48.6 | 67.8 |
| Cycle 2 | 40.0 | 7.7 | 21.5 | 71.3 | 13.4 | 52.1 | 85.9 | 44.0 | 65.0 |
| Cycle 3 | 25.6 | 0.8 | 12.2 | 64.8 | 18.4 | 43.2 | 72.5 | 31.5 | 52.7 |

Cattle pen enclosures

| | | | | | | | | | |
|---|---|---|---|---|---|---|---|---|---|
| Cycle 1 | 40.4 | 9.0 | 21.2 | 94.4 | 19.5 | 72.7 | 85.5 | 51.9 | 69.1 |
| Cycle 2 | 40.0 | 6.4 | 19.3 | 98.7 | 23.1 | 79.2 | 83.3 | 48.3 | 65.5 |
| Cycle 3 | 26.6 | 0.8 | 11.5 | 99.9 | 27.5 | 86.8 | 70.2 | 38.7 | 35.2 |

¹The temperature (TA) and relative humidity (RH) within CFE were monitered every 15 s during the 15-min emissions sampling periods using RH/T sensors (Dwyer instruments, Inc., Michigan City, IN), and the same measurements were obtained for outside ambient air continuously using an on-site weather station (Novalynx, Model 110-WS-16, Auburn, CA.).
²THI was calculated using the equation of Mader et al. (2006) where THI = (0.8 × TA) + |(RH × 0.01) × (TA − 14.4)| + 46.4; TA = ambient temperature; RH = relative humidity %.

### Health Observations

Cattle were observed daily by trained personnel and abnormal health observations were recorded. Health conditions observed that would deem an animal ineligible or potentially require removal later in the study were documented to prevent affected cattle from being considered for study enrollment. Additional observations were performed by a licensed veterinarian as cattle progressed through the marketing channel, including during loading onto the semi-trailers, during unloading at the abattoir, and finally as ante-mortem observations after a minimum lairage time of 5 h. All cattle euthanized or found dead were necropsied by a veterinarian. During the treatment period, six animals were found dead, and four others were removed from the study due to various conditions.

### Diet Formulation and Feed Assays

Cattle had been fed a concentrate-based diet in a commercial setting before arrival at the study site on day −28, at which point they were provided ad libitum access to water and re-acclimated to a concentrate-based diet using a step-up program involving two intermediate diets based on increasing concentrate levels (˜60% and 70%, respectively) and varying proportions of alfalfa and wheat hay. On day −14, cattle were transitioned onto a finishing diet (Table 1) formulated to meet, or exceed, the minimum nutrient requirements for growing beef cattle (NRC, 2000) that was then fed for the remainder of the study. A nonmedicated supplement (i.e., type B feed with a ground corn carrier) was included as 2.5% of the diet DM during acclimation for all cattle from day −14 until the beginning of treatment administration. On day 0, one of four type B supplements were added to the basal diet to provide either 0.0 (CON), 1.38, 5.5, or 22.0 mg·kg⁻¹ (DM basis) LUB in a type C (i.e., final feed) medicated feed. All type C feeds containing the appropriate concentrations of LUB or CON were prepared at the study site by adding the same proportions of Type B supplement, water, and basal ration in a rotary mixer wagon (Roto-Mix Forage Express, Dodge City, KS). Mixer procedures (i.e., LUB potency, homogeneity) were validated (i.e., CV≤15%) before study initiation. No concomitant feed additives (ionophores, antibiotics, estrous suppressors, and β-agonists) were used at any point during this study. The mixer wagon was cleaned between each treatment using a flush load comprised of straw and water. Cleaning was also validated to ensure there was no carry-over of LUB between batches. The digital scale on the mixer wagon measured feed deliveries with a 0.45-kg resolution. Prior to use each day, the scale was verified using certified check weights.

Target nutrient densities (% of DM) for CP (13.5%), Ca (0.7%), and P (0.3%) were set based on the recommendations of consulting feedlot nutritionists reported in a survey by Vasconcelos and Galyean (2007). Triplicate samples were collected daily during delivery from the mixer wagon into the bunk for each batch of complete feed and frozen until analysis. Three of the seven composite samples representing a week were randomly selected for each treatment and combined and subsampled for weekly analyses of nutrient content [AOAC methods #985.01 (Ca and P) and 990.03 (CP: Minnesota Valley Testing Laboratories, New Ulm, MN] and LUB concentration (Covance Laboratories, Inc., Greenfield, IN). The minimum acceptable assay value for Ca (0.3%) and P (0.2%) was set to the NRC minimum nutrient requirement for growing beef cattle (NRC, 2000), whereas the minimum acceptable value for CP was set at 12.5%. The threshold for CP was chosen as this was the minimum level recommended by feedlot nutritionists (Vasconcelos and Galyean, 2007). No samples fell below the assay thresholds for CP, Ca, or P.

LUB concentrations were required to be within +25% of the target for the 1.38 and 5.5 mg·kg⁻¹ samples, and ±20% for the 20 mg· kg⁻¹ samples in accordance with FDA guidance (FDA, 2012). The mean LUB potency values for each weekly composite sample over all three study cycles were within the acceptable assay concentration range for each dose level (results not shown). Finally, feed samples from CON were assayed for LUB to confirm the mixer wagon cleaning procedure prevented feeding of LUB, and levels were below the level of quantification (LOQ=0.2 g/ton) in each sample assayed.

### Feeding and Growth Performance

Individual unshrunk BW measurements were obtained prior to feeding using a certified scale with a 0.45-kg resolution on day −8 (randomization), 0 (initial BW), 7, 14, 28, 56, and 91 d (final BW). Additionally, prior to use the scale was verified with check weights. Feed bunks were assessed daily for each CPE by trained personnel, who estimated orts from the previous day and determined the amount to be provided in a single delivery to ensure ad libitum access to feed. Orts remaining on day 91 were weighed to adjust for refused feed, and DMI (kg·animal⁻¹·d⁻¹) was calculated by dividing the total feed delivery less refused feed by the cumulative number of cattle-days in the CPE to determine as-fed consumption, and then multiplying by diet DM. Thus, cattle were removed from treatment diets a minimum of 24 h before slaughter in order to comply with the Food Use Authorization (FDA, 2016) granted by the FDA. The CPE mean for unshrunk initial and final BW were used to calculate ADG over the 91-d period, and G:F was calculated as a quotient of ADG divided by DMI.

### Slaughter, Carcass Measurements, and Meat Quality

On day 91, cattle were loaded onto double-decked aluminum semi-trailers and transported ˜1,000 km to a commercial abattoir where they were slaughtered following an ˜5 to 9 h lairage. Carcass identification was maintained throughout the slaughter process by recording ear tag sequence at stunning and then cross-matching to sequentially numbered carcass tags. Hot carcass weights and KPH were measured following industry standard processing, and yield grade (YG) and quality grade data were collected from left carcass sides by trained university personnel after 22 h in a spray-chill system.

Following chill, striploins (LM) were collected from three randomly selected cattle/CPE and shipped to the University of Illinois Meat Science Laboratory for Warner-Bratzler shear force (WBSF) determination. At the laboratory, the anterior end of the striploin was fabricated into 2.54-cm steaks, vacuum packaged, and aged at 4° C. until 14-d postmortem. Steaks were frozen after aging, and then thawed at 4° C. for 24 h before being cooked on a Farberware Open Hearth electric broiler (Farberware, Bronx, NY). Copper-constantan Type-T thermocouples (Omega Engineering. Stamford, CT) connected to a digital scanning thermometer (Barnant Co., Barington, IL) were used to monitor internal temperature, and each steak was flipped a single time when the internal temperature reached 35° C. The steaks were removed from the grill when a temperature of 70° C. was achieved and cooled to ˜25° C. before six cores (1.25 cm diameter) were removed parallel to muscle fiber orientation. Cores were sheared perpendicular to the muscle fibers using a Texture Analyzer TA.HD Plus (Stable Microsystems, Godalming. UK) equipped with a WBSF attachment, and the peak WBSF measurement was averaged over all 6 cores to obtain a single shear force measurement (kg of force) for each steak.

### Statistical Analysis

Data were analyzed using version 9.2 of SAS (SAS Institute, Cary, NC), and the individual CPE were considered the experimental unit. Continuous variables were analyzed using PROC MIXED, with treatment (LUB dose), sex, and the dose×sex interaction as fixed effects, and cycle as the random effect included in the model. If the dose×sex interaction was not significant (P>0.05), the main effect of dose pooled across sexes was evaluated. When the main effect of dose was significant (P≤0.05) or tended to be significant (0.05≤P≤0.10), planned contrasts comparing each LUB dose to CON were performed in a pairwise fashion. If the dose×sex was significant (P≤0.05) planned contrasts comparing each LUB dose to CON within sex were performed in a pairwise fashion. Treatment means were estimated using the LSMEANS statement.

Prior to study conduct, the CVM concurred that for the claim variables (gas emissions per unit of BW or HCW) the dose range for each variable would include those dosages significantly different from CON. Additionally, methodology to determine the minimum effective dosage and maximum effective dosage was agreed upon. The minimum effective dose for the claim variables was determined to be the smallest dose used in the study that differed from CON based on the planned contrasts performed following a significant F-test (P<0.05). To determine the lowest maximum effective dose, a dose-response curve fit to the least squares means of the doses was performed. If the dose-response curve was determined to be a linear plateau model (Anderson and Nelson. 1975) and the slope or slopes were different (P<0.05) from zero, then the maximum effective dosage was the “join point” where the plateau began. The join point was identified by assessing five specific, competing linear and linear plateau models based on the smallest P-value indicating best fit: (i) Linear=linear from 0 to 22.0 mg·kg⁻¹ DM: (ii) Quadratic1=linear from 0 to 1.38 mg·kg⁻¹ DM, plateau from 1.38 to 22.0 mg·kg⁻¹ DM: (iii) Quadratic2=linear from 0 to 5.5 mg·kg⁻¹ DM, plateau from 5.5 to 22.0 mg·kg⁻¹ DM: (iv) Quadratic3=no response from 0 to 1.38 mg·kg⁻¹ DM, but linear from 1.38 to 5.5 mg·kg⁻¹, plateau from 5.5 to 22.0 mg·kg⁻¹ DM; and (v) Quadratic4=no response from 0) to 1.38 mg·kg⁻¹, and linear from 1.38 to 22.0 mg·kg⁻¹ DM.

Discrete variables were analyzed with a generalized linear mixed model using a binomial distribution and logit link function in PROC GLIMMIX. The classification of fixed and random effects and the handling of interactions and pairwise comparisons were performed in a similar manner as the continuous variables. Statistical analyses for YG were performed on both continuous and discrete (YG 1=1.00 to 1.99, YG 2=2.00 to 2.99, and so on) forms of data, and quality grades were further sorted into the five categories routinely used for determining premium or discount adjustments when cattle are marketed on a grid-based system (U.S. Prime, upper 2/3 Choice, low Choice, Select, and Standard). Because the model did not converge due to sparseness of data for mortality, YG 4, and Select and Prime quality grades. Fisher's exact test was performed using PROC FREQ to evaluate the frequency distribution of the CON cattle compared with LUB. Statistical significance for the main effects of dose was determined by P≤0.05 and tendencies were declared when 0.05≤P≤0.10.

### Results

There were no interactions observed between dose and sex for any variable measured in the study (P≥0.063).

### Growth Performance and Carcass Characteristics

Initial BW did not differ (P=0.937) among LUB treatments, and there was no effect (P=0.585) of LUB on DMI (Table 6). Compared with cattle receiving CON, G:F was increased (P≤0.065) by 8.3%, 9.7%, and 13.2% for cattle fed 1.38, 5.5, and 22.0 mg·kg⁻¹ LUB, respectively. The effect of LUB treatment on G:F was reflected by a tendency to improve (P=0.075) ADG, with ADG increased (P≤0.067) vs. CON by 11.8%, 9.4%, and 12.6% in cattle fed 1.38, 5.5 and 22.0 mg·kg⁻¹ of LUB, respectively: yet final BW was not altered by LUB treatment (P=0.257).

Compared with CON cattle, cattle fed LUB had carcass weights about 15 kg heavier (P≤0.035), dressing percentages 0.9 to 1.3 units greater (P≤0.006), and LM areas 6.4 to 8.4 cm² larger (P≤0.001; Table 3). Adjusted fat thickness was not affected (P=0.579) by LUB treatment. Compared with carcasses from CON cattle, KPH was only reduced (P=0.001) when cattle were fed 22.0 mg·kg⁻¹ LUB (1.96% vs. 1.61%; Table 3). LUB treatment had no effect (P=0.155) on calculated YG. When YG was analyzed as a discrete variable, the probability of cattle producing a YG3 carcass was greater (P<0.019) for CON-than LUB-fed cattle: the probability of cattle producing YG1, YG2, or YG4 carcasses was similar (P≥0.233) across treatments.

LUB treatment had no effect (P≥0.262) on skeletal, lean, or overall maturity. Marbling scores tended (P=0.058) to be influenced by LUB treatment with carcasses of LUB-fed cattle having marbling scores 50 to 63 points less (P≤0.051) than CON carcasses (Table 3). Feeding LUB shifted the quality grade distribution lower, whereby cattle fed LUB had a lower (P=0.004) probability of grading high Choice and a greater (P=0.021) probability of grading low Choice than CON. Dark cutter incidence was not influenced by treatment, as only a single carcass fell into this category over the entire study. WBSF was 0.27 to 0.44 kg greater (P≤0.039) for striploins from LUB-treated cattle than CON cattle (Table 3).

TABLE 3
Least squares means for the effect of lubabegron (LUB) dose on growth performance
traits and carcass characteristics of beef cattle over a 91-d treatment period

| | | | | | | | | Significance of contrast | | |
|---|---|---|---|---|---|---|---|---|---|---|
| | | | | | | | | Control vs. | Control vs. | Control vs. |
| | LUB (mg · kg⁻¹ DM) | | | | | Dose × sex | Dose | 1.38 mg · | 5.5 mg · | 220 mg · |
| Variable | 0.0 | 1.38 | 5.5 | 22.0 | SEM | P-value¹ | P-value¹ | kg⁻¹ | kg⁻¹ | kg⁻¹ |

Growth performance²

| | | | | | | | | | | |
|---|---|---|---|---|---|---|---|---|---|---|
| Unshrunk initial BW, kg | 451 | 454 | 455 | 452 | 10.0 | 0.996 | 0.937 | | | |
| Unshrunk final BW, kg | 567 | 583 | 582 | 582 | 19.4 | 0.916 | 0.257 | | | |
| DMI, kg | 8.8 | 9.2 | 8.8 | 8.8 | 0.56 | 0.980 | 0.585 | | | |
| ADG, kg | 1.27 | 1.42 | 1.35 | 1.43 | 0.126 | 0.724 | 0.075 | 0.027 | 0.067 | 0.023 |
| G:F, kg:kg | 0.144 | 0.156 | 0.158 | 0.163 | 0.0071 | 0.856 | 0.031 | 0.065 | 0.033 | 0.005 |

Carcass characteristics

| | | | | | | | | | | |
|---|---|---|---|---|---|---|---|---|---|---|
| HGW, kg | 349 | 364 | 365 | 365 | 11.7 | 0.865 | 0.035 | 0.019 | 0.014 | 0.014 |
| Dressing percentage² | 61.5 | 62.4 | 62.7 | 62.8 | 0.34 | 0.738 | 0.002 | 0.006 | <0.001 | <0.001 |
| Adjusted fat thickness, cm | 1.28 | 1.15 | 1.24 | 1.19 | 0.119 | 0.994 | 0.579 | | | |
| IM area, cm² | 88.4 | 94.8 | 06. 1 | 96.8 | 1.78 | 0.063 | <0.001 | <0.001 | <0.001 | <0.001 |
| Marbling score³ | 623 | 573 | 560 | 562 | 16.7 | 0.979 | 0.058 | 0.051 | 0.018 | 0.022 |
| Calculated yield grade⁴ | 2.68 | 2.37 | 2.41 | 2.25 | 0.154 | 0.816 | 0.155 | | | |
| KPH % | 1.96 | 1.99 | 1.96 | 1.61 | 0.066 | 0.802 | 0.002 | 0.742 | 0.981 | 0.001 |
| Lean maturity⁵ | 162 | 163 | 167 | 163 | 3.5 | 0.731 | 0.262 | | | |
| Skeletal maturity⁵ | 172 | 170 | 170 | 172 | 1.9 | 0.585 | 0.592 | | | |
| Overall maturity⁵ | 169 | 168 | 169 | 168 | 2.5 | 0.639 | 0.805 | | | |
| 14-dWBSP, kg | 2.48 | 2.79 | 2.92 | 2.75 | 0.117 | 0.620 | 0.017 | 0.022 | 0.003 | 0.039 |

¹If the dose × sex interaction was not significant. (P > 0.05), contrasts were constructed to evaluate the main effect of dose for the pooled sexes. Statistical significance for dose was declared when P < 0.05 and tendencies were declared when 0.05 < P ≤ 0.10.
²Growth performance and dressing percentage were based on unshrunk initial and final BW.
³500 [symbol] , Small [symbol] . 600, Modest [symbol] .
⁴Yield Grade = 7.50 + (0.98 × adj. fat thickness, cm) + (0.2 × KPH, %) + (0.0084 × HCW, kg) − (0.05 × LM, cm) (USDA, 1997).
⁵100, A [symbol] maturity; 200, B [symbol] maturity.
⁶WRSF: Warner- [symbol] shear force, measured after a 14-d aging period. [symbol]
indicates data missing or illegible when filed

### Sex Effects

Steers started the treatment period 43 kg heavier (P<0.001) than heifers. During the 91-d period, steers consumed 0.5 kg/d more DM (P=0.038) than heifers. Both sexes gained weight similarly (P=0.875), resulting in heifers tending to have 5% greater G:F (P=0.064) than steers (Table 4). Heifers produced carcasses that were 30 kg lighter (P<0.001) than steers with 1.17 cm greater (P=0.024) adjusted fat thickness and 0.45% units greater (P<0.001) KPH; LM area and calculated YG did not differ (P>0.216) between steers and heifers. Lean maturity was not affected (P>0.854) by sex, but heifers had marginally higher (P<0.001) skeletal maturity (A75 vs. A67) than steers. Marbling score and WBSF did not differ (P>0.142 Increased HCW for each of the respective LUB treatments was 15, 16, and 16 kg animal-1. The theoretical efficiency of N conservation was 69.9%, 55.7%, and 49.9% for 1.4, 5.5, and 22 mg·kg⁻¹ DM LUB, respectively.

TABLE 4
Least squares means for the effect of sex on growth performance, carcass
characteristics, and NH3 gas emission in beef cattle over a 91 d period

| | Sex | | | Dose × sex | Sex |
|---|---|---|---|---|---|
| Variable | Steers | Heifers | SEM | P-value [symbol] | P-value [symbol] |
| Growth performance [symbol] | | | | | |
| Unshrunk initial BW, kg | 475 | 432 | 9.2 | 0.996 | <0.001 |
| Unshrunk final BW, kg | 601 | 556 | 18.9 | 0.918 | <0.001 |
| DMI, kg | 9.1 | 8.6 | 0.53 | 0.980 | 0.038 |
| ADG, kg | 1.38 | 1.38 | 0.12 | 0.724 | 0.875 |
| G [symbol] F [symbol] kg [symbol] kg | 0.151 | 0.159 | 0.0065 | 0.856 | 0.064 |
| Carcass characteristics | | | | | |
| H [symbol] W, kg | 376 | 346 | 11.3 | 0.865 | <0.001 |
| Dressing percentage [symbol] | 62.5 | 62.2 | 0.30 | 0.738 | 0.078 |
| Adjusted fat thickness, cm | 1.13 | 1.30 | 0.110 | 0.994 | 0.024 |
| LM area, cm [symbol] | 14.7 | 14.5 | 0.26 | 0.063 | 0.216 |
| Marbling score [symbol] | 567 | 592 | 11.8 | 0.979 | 0.156 |
| Calculated yield grade [symbol] | 2. [symbol] | 2.46 | 0.137 | 0.816 | 0.582 |
| KPH, % | 1.65 | 2.10 | 0.049 | 0.802 | <0.001 |
| Lean maturity [symbol] | 164 | 164 | 3.3 | 0.731 | 0.854 |
| Skeletal maturity [symbol] | 167 | 175 | 1.7 | 0.585 | <0.001 |
| Overall maturity [symbol] | 166 | 171 | 2.4 | 0.639 | <0.001 |
| 14-d WBSF [symbol] , kg | 2.80 | 2.67 | 0.100 | 0.620 | 0.142 |

[symbol]

If the dose × sex interaction was not significant (P > 0.05), the main effect of sex was evaluated for the pooled doses. Statistical significance for sex was declared when P ≤ 0.05 and tendencies were declared when 0.0 [symbol] ≤ P ≤ 0.10. [symbol]
Growth performance and dressing percentage were based on unshrunk initial and final BW. [symbol]
500, Small [symbol] 600, Modest [symbol] . [symbol]
Yield Grade = 2.50 + (0.98 × adj. fat thickness, cm) [symbol] (0.2 × KPH, %) + (0.0084 × HCW, kg) − (0.05 × LM, cm [symbol] ) (USDA, 1987). [symbol]
100, A [symbol] maturity; 200, B [symbol] maturity. [symbol]
WBSF, Warner-Bratzler shear force, measured after a 14-d aging period. [symbol]
indicates data missing or illegible when filed

In addition to the increase in carcass weight discussed above, evaluation of other carcass characteristics provided further information on the physiological effects of LUB. Routine measurements of carcass fat showed no change to adjusted fat thickness, a decrease in KPH at only the highest LUB dose, and a reduction in marbling score for all doses of LUB. In the current study, cattle that received LUB had a 7% to 10% increase in LM area and a corresponding 8% to 10% decrease in marbling score.

Cattle that received LUB for 91 d produced LM steaks with mean 14-d WBSF values that were 0.27 to 0.44 kg greater than that of CON. Little comparative data are available for shear force of steaks from LUB-fed cattle, however, increases in shear force are not unexpected as similar effects have been reported for other biotechnologies that alter muscle development (Garmen and Miller, 2014). Despite an increase in WBSF, mean shear force values of steaks from LUB-treated cattle in the current study (2.75 to 2.92 kg) were within the range of recent North American surveys that report WBSF for top loin steaks (˜2.0 to 3.4 kg Guelker et al., 2013; Howard et al., 2013; Igo et al., 2015; Martinez et al., 2017). The influence of changes in shear force on consumer acceptability have been reported to vary depending on where within the range of WBSF observations occur (Platter et al., 2003), thus additional evaluations of shear force for LUB-fed cattle would further the understanding of this effect. The present study measured shear force at a single postmortem aging period of 14 d, however, the most recent National Beef Tenderness Survey (Martinez et al., 2017) reported post-fabrication aging time for boneless striploins or toploins was 27.2 d for retail stores and 34.6 d for foodservice operations (Martinez et al., 2017).

### Example 2: Effects of Various Doses of Lubabegron on Growth Performance, and Carcass Characteristics of Beef Cattle During the Last 56 Days of the Feeding Period

This example describes a randomized complete block study that evaluated LUB dose (0, 1.5, 3.5, and 5.5 mg kg⁻¹ dry matter) during the last 56 d of the feeding period on live weight, carcass weight, and associated ratios in beef feedlot cattle. Carcass characteristics, mobility, and health were also evaluated. All cattle received monensin and tylosin throughout the study.

This study was conducted at Cactus Research, Amarillo, TX, USA. Animal care and disposal methods were in accordance with applicable Federal, State, and Local regulations. All study procedures were reviewed and approved by Elanco's Institutional Animal Care and Use Committee (IACUC; Approval number EIAC-0827).

### Experimental Design and Treatments

A randomized complete block design was used to evaluate the effects of LUB on calculated growth performance of 2,880 British and Continental European crossbred steers typical for U.S. feedlots. Bos indicus breeding was limited to less than ⅛. The study evaluated four

LUB doses: 0 (control, CON), 1.5, 3.5, and 5.5 mg·kg⁻¹ DM. There were 12 blocks, each block containing four replicate pens that started LUB feeding on the same date. Steers were randomized within each block of four pens with 60 steers per pen, resulting in a total of 720 steers enrolled in each treatment. LUB feeding started on Jun. 24, 2018, Jul. 1, 2018, and Jul. 15, 2018, four blocks on each date.

### Study Timeline and Treatment Allocation

On or prior to d −85 (i.e., 85 days prior to slaughter) and before randomization, animals eligible for enrollment were sorted based on BW, days on feed, phenotype, health, and general disposition, with the most uniform approximately 1,000 steers selected for potential enrollment in each time replicate group. Animals were examined by a qualified evaluator and screened for eligibility based on the following inclusion criteria: confirmed to be a steer and determined to be in good health. Steers received a Revalor XS implant (trenbolone acetate and estradiol; Merck Animal Health, Summit, NJ) 4.7 to 7.0 months before slaughter (i.e., d 0). Animals with existing or pre-existing abnormal health conditions or observations were eligible for inclusion in the study if the condition was considered to be minor in nature (e.g., minor lacerations, eye redness, dermatitis, etc.) and not expected to worsen or impact the animal's ability to grow normally and complete the study. Animals that appeared unthrifty, ill, or injured were excluded. In addition, animals were excluded due to extreme BW.

Randomization of animals to pens occurred 66 to 69 d before slaughter, which was considered the start of the approximately 10 to 15 d acclimation phase. The mean BW of the 12 blocks ranged from 538.4 to 570.6 kg. The minimum and maximum within-block BW range across all 12 blocks were 106.1 and 118.8 kg, respectively. These data suggest the cattle were uniform across blocks and within each block to ensure acceptable uniformity of cattle at harvest (slaughter). Cattle were assigned into one of four blocks during each randomization event (i.e., time replicate, n=3). For each time replicate, there were four blocks of four pens (one pen for each dose level). Allotment of animals to pens was conducted 9 to 12 d prior to the first feeding of experimental diets. The experimental diets were fed for 56 d. Complete blocks (four pens, one pen per treatment group) were allocated on a given day. Pens contained 60 steers each and were contiguous in proximity. The randomization schedule was prepared using a Microsoft Excel spreadsheet. At the time of randomization, steers were individually weighed and assigned to study pens if within target weight range of +56.7 kg. Each succeeding group of four candidate steers was placed into one of four pens within the block using the randomization schedule. Subsequent blocks were populated by repeating the same procedure. A separate randomization schedule was used to assign one of the four treatments to each of the four pens in each block.

### Health Observations

During the approximately 10 to 15 d acclimation phase and the entire treatment phase until cattle were loaded for slaughter, all animals were observed by trained caretakers at least once daily, but health issues were only noted by exception. All abnormalities were recorded even if considered common for feedlot cattle. Abnormal health observations were observations that the observer considered as 1) typical for beef cattle at that age, or 2) not causing undue pain or distress to the animal, and/or, 3) not impeding the animal's growth at the time the observation was made. Animals with abnormal health observations were allowed to remain in the study. Animals were allowed to receive concomitant therapies (e.g., antibiotic treatment for respiratory disease and treatment for bloat). Health observations requiring an animal to be classified as “removed” were abnormalities that the observer considered as 1) resulting in pain or distress to the animal, or 2) likely to result in further deterioration of the animal's health, and/or 3) impairing the animal's ability to access food or water at the time of the observation. Animals removed from the study during the treatment or withdrawal phase were penned separately in the research feedlot and tracked through slaughter to assure accountability of all cattle on study per food use authorization or were euthanized and necropsied. Decisions to remove an animal from treatment phase were made by the Investigator or Manager. Animals that were found dead at the time of the observation were removed from the pen as soon as practical and necropsied (if possible).

### Diet Formulation and Feed Assays

Beginning no later than approximately d −70, animals were fed a diet containing Rumensin (monensin 46.3 mg·kg^{-1 100}% DM basis; Elanco, Greenfield, IN, USA) and Tylan (tylosin 8.9 mg·kg^{-1 100}% DM basis; Elanco) ad libitum. Cattle in the LUB treatments were switched from the basal finishing ration to the finishing ration containing the appropriate concentrations of LUB Type A premix at the start of the treatment period (d −57). Table 5 lists the composition and the formulated nutrient composition of the finishing diet. The diet was designed to meet or exceed the minimum nutrient requirements cited in Nutrient Requirements of Beef Cattle (NASEM, 2016). LUB was delivered to the cattle using a 1% LUB Type A premix prepared and delivered by Elanco (Clinton Laboratories, Clinton, IN, USA), and the ration was prepared on-site at the research facility. The 1% Type A premix was delivered into the ration by flushing the 1% Type A premix through a micro-ingredient machine (Micro Technologies, Amarillo, TX) with water, which was included at approximately 27 kg of the “as-fed” ration, for each 3,629 kg batch. The 1% Type A premix was included in the finishing ration at 0, 0.0154%, 0.0353%, or 0.0551% (100% DM) to provide CON, 1.5, 3.5, or 5.5 mg·kg⁻¹ LUB (100% DM). The on-site feed mixers used to mix (stationary horizontal paddle mixer [Cactus Varied Industries, Amarillo, TX, USA]) and deliver (Roto-Mix 490-14 and Roto-Mix 620-16, Roto-Mix LLC, Dodge City, KS) the Type C feed were qualified to confirm that the ration could be mixed homogeneously before feeding medicated feed. Cattle were fed in order of CON, 1.5, 3.5, or 5.5 mg·kg⁻¹ doses of LUB. Prior to mixing the CON batches, the mixer was flushed with appropriate amount of non-medicated feed or other cattle feed to assure no carryover of test compound. Feed was issued three times each day at approximately the same time each day. Bunk calls were made at the time of first feeding and amount of feed issued for the third feeding was adjusted based on daily bunk calls according to site procedures. The feeding goal was to have slick bunks prior to the first feeding and approximately ⅓ of the cattle at the bunk, ⅓ of the cattle moving to the bunk, and ⅓ of the cattle not moving to the bunk at time of first feeding. Water was available ad libitum throughout the study. The weight of feed issued was recorded and electronic feed records were provided for data analysis.

TABLE 5
Ingredient composition, analyzed nutrient content, and the formulated
nutrient and monensin/tylosin composition of the finishing
diet fed during the acclimation and treatment phases

| Ingredient composition, % dry matter (DM) [symbol] | |
|---|---|
| Flaked corn | 57.3 |
| Wet distillers grains | 17.3 |
| Sweet [symbol] plus [symbol] | 17.0 |
| Cotton [symbol] | [symbol] |
| Corn oil | 1.3 |
| Water plus micro-ingredients | Approximately 0.05 |
| Total | 100.0 |
| Formulated nutrient and [symbol] / [symbol] concentration | |
| DM | 63.1 |
| Crude protein (CP), % of DM | 14.6 |
| Degradable intake protein, % of DM | 7.8 |
| Crude protein equivalent from non-protein nitrogen (NPN), | 0.8 |
| % of DM tom non-protein nitrogen % of DM | |
| Ether extract, % of DM | 5.7 |
| Neutral detergent fiber (NDF), % of DM | 19.9 |
| Physically effective NDF, % of DM | 11.2 |
| Ca, % of DM | 0.8 |
| P, % of DM | 0.5 |
| K, % of DM | 0.7 |
| Mg, % of DM | 0.2 |
| Sa [symbol] , % of DM | 0.5 |
| S, % of DM | 0.2 |
| Co, mg [symbol] kg of DM | 0.5 |
| Cu, mg [symbol] kg [symbol] of DM | 13 |
| [symbol] mg [symbol] kg [symbol] of DM | 0.5 |
| Fe, mg [symbol] kg [symbol] of DM | 84 |
| Mn, mg [symbol] kg [symbol] of DM | 45 |
| [symbol] mg [symbol] kg [symbol] of DM | 0.36 |
| Zn, mg [symbol] kg [symbol] of DM | 79 |
| Calculated NE [symbol] of DM | 2.15 |
| Calculated NE [symbol] of DM | 1.49 |
| Calculated [symbol] mg [symbol] kg [symbol] of DM | 46.3 |
| Calculated [symbol] mg [symbol] kg [symbol] of DM | 8.9 |

[symbol]

The mean of wet chemistry analysis (DM [symbol] ) of 11 weekly ration samples collected during the treatment phase of the study for each of the four treatment groups was 15. [symbol] % CP, 0.86% Ca, 0.48% P, 1.2% NPN, 2.0% acid-detergent insoluble nitrogen (ADIN). The range of mean CP for the four treatment groups was 15.4% to 15.7%. Samples analyzed by Servi-Tech Laboratories (Amarillo, TX). [symbol]
Sweet [symbol] wet corn gluten feed (Cargill Corn Milling, [symbol] TX) with added (as fed) calcium carbonate (4%), salt (1.8%), urea (1.1%), and trace mineral premix (0.2%). [symbol]
Water was included at 27.2 kg per batch through the micro-ingredient machine for inclusion of [symbol] mon [symbol] and [symbol] Each batch of manufactured feed weighed approximately 3 [symbol] kg. [symbol]
Formulated to provide the following in each kilogram of total diet DM: 2,645 IU Vitamin A, 265 [symbol] U Vitamin D, 46.3 mg [symbol] .8.9 mgtylosin, and CON, 1.5, 3.5, or 5.5 mg of [symbol] [symbol]
Calculated based on NASEM (2016). [symbol]
Provided at approximately 464 mg [symbol] [symbol] day [symbol] [symbol]
Provided at approximately 90 mg [symbol] day [symbol] [symbol]
indicates data missing or illegible when filed

Samples (separate samples for LUB and nutrient analysis) were collected weekly from each treatment of ration prepared (d −57 to d −1) for analysis. Duplicates consisting of a composite of three samples from different locations within the delivered truck load were used for either a primary or a back-up sample. The weekly primary samples were analyzed for LUB at Eurofins Laboratories (Greenfield, IN). Back-up samples were frozen. Feed assays were performed using a validated analytical method for LUB (Determination of Lubabegron in Medicated Feed by High-Performance Liquid Chromatography: Laboratory Procedure G1635). The permissible analytical variation of LUB content in a single feed analysis (weekly composite) was ±25% for the 1.5 mg·kg⁻¹ truck-loads and ±20% for the 3.5 and 5.5 mg·kg⁻¹ truck-loads.

Samples for nutrient analysis were submitted fresh on the day the samples were collected to Servi-Tech Laboratories, Inc. (Amarillo, TX). Analysis included DM (National Forage Testing Association procedure #2.1.4 Dry Matter by Oven Drying for 3 hr at 105° C.), CP (AOAC #990.03), N (% CP-6.25), non-protein nitrogen (NPN) (AOAC #941.04), acid detergent insoluble nitrogen (ADIN) (AOAC #2001.11), and Ca and P (AOAC #990.08).

### Feeding and Growth Performance

Animals were weighed individually at the time of randomization (approximately d −69 to d −66) and this weight was used to exclude steers with extreme BW from the trial. All other BW (unshrunk) were collected by pen and used for calculation of growth performance. Scheduled pen weights were taken prior to feed delivery and collected on d −59 (2 d before treatment start, which was considered the treatment start weight) and d 0 (1 d after the end of treatment, which was considered the treatment end weight). Prior to each collection of animal weights, the scales were qualified following site operating procedures.

Feed weighback (if feed remained in bunk) occurred on d −57, d −1, d 0, and whenever required due to soiled, spoiled or excessively wet feed. When feed weighbacks occurred between d −59 and d 0, a composite sample of weighback from all bunks was collected and DM determined on each sample on-site. Average daily DMI was calculated by subtracting weighbacks from the amount offered the previous day as-fed and then multiplying intake as-fed by the dietary DM and adjusting for total animal-days during the treatment period. Average daily gain (ADG) (kg·hd⁻¹) was calculated for the entire 56 d treatment period using unshrunk BW (kg) excluding the animals removed during the treatment period. Gain efficiency was summarized as the gain:feed quotient (ADG:DMI).

### Animal Mobility

Animal mobility assessments were conducted three times prior to harvest at: 1) approximately 1 week prior to harvest while cattle were in their home pens; 2) after collection of final pen weights and immediately prior to loading into trucks for slaughter; and 3) at antemortem inspection during lairage at the packing plant. The mobility scorer used the North American Meat Institute's (NAMI) mobility scoring system (Edwards-Callaway et al., 2017) to evaluate cattle mobility. The NAMI mobility scoring system is a 4-point scoring system where 1=normal, walks easily with no apparent lameness or change in gait; 2=keeps up with normal cattle when the group is walking, exhibits one or more of the following: stiffness, shortened stride, or slight limp; 3=lags behind normal cattle when the group is walking, exhibits one or more of the following: obvious stiffness, difficulty taking steps, obvious limp, or discomfort; 4=extremely reluctant to move, even when encouraged by handlers.

### Slaughter and Carcass Characteristics

LUB was removed from feed at least 24 h before harvest to comply with the Food Use Authorization (FDA, 2016) granted by the FDA. Cattle were loaded for harvest shortly after collection of final pen live weights and transported to the slaughter facility at a stocking density of approximately 30 steers (i.e., half a pen of 60 steers) in one semi-trailer. The slaughter process was conducted in accordance with USDA requirements and standard slaughter site procedures. Animal/carcass identification was maintained by sequentially recording the ear tag numbers of cattle as they were slaughtered via a sequence number affixed to each carcass by trained personnel from the Beef Carcass Research Center (BCRC; West Texas A&M University, Canyon, TX) and correlating the individual ear tag number to the plant-assigned carcass ID number, which remained intact throughout the carcass data/sample collection phase. Following an industry-standard dressing procedure, each carcass was weighed for determination of HCW.

Carcass evaluation occurred following the commercial plant's standard chilling period. The carcass quality and yield grade factors, marbling score, ribeye area, and objective 12th rib fat thickness were captured objectively using the VBG2000 camera system. Additionally, adjusted 12th rib fat thickness, kidney, pelvic, and heart (KPH) fat assessment, and other defects (dark cutters, blood splash, etc.) were recorded by trained personnel (BCRC). Skeletal maturity was obtained only on carcasses that were “B” maturity or greater, otherwise, skeletal, lean, and overall maturity and lean color were not obtained.

Carcass defects (dark cutters, excessive trim) were noted by exception. The objective fat thickness was used to calculate the USDA yield grade. All carcasses were considered to be less than 30 mo of age; therefore, USDA quality grade was determined by using marbling score (USDA, 2019). The severity of the dark cutting condition was indicated by assigning the dark cutting carcasses to ⅓, ⅔, or full dark categories. Dark cutters were not excluded when determining quality grade, however, a summary of the frequency of dark cutter carcasses by treatment group is provided. Carcasses identified as excessively trimmed (greater than approximately 9.1 kg trim) were not excluded from the analysis, however, a summary of the frequency of excessively trimmed carcasses by treatment group is provided.

### Statistical Analysis

The label claim variable of cumulative NH₃ gas emissions over the treatment period normalized by final BW (with d −59 BW as a covariate) for the period and HCW (g of gas kg⁻¹ of BW or g of gas kg⁻¹ HCW) was determined by utilizing NH₃ gas emissions equations developed from the LUB clinical effectiveness study (FDA-FOI, 2018) and validated using animals of similar BW as described by Brown et al. (2019). The pen was the experimental unit for each outcome. Differences were deemed significant using a two-sided test at Pₜᵣₜ≤0.05, however, a less stringent significance threshold of Pₜᵣₜ≤0.10 was used for abnormal health observations. Fixed effect of treatment and random effects of time replicate and block within time replicate were included in the model. Discrete variables were analyzed using a generalized linear mixed model, Proc GLIMMIX SAS version 9.4 unless there were convergence problems due to sparseness of data. A binomial distribution was assumed and a logit link used in the analysis. Contrasts were constructed between the CON group and each non-CON dosage group, and differences were deemed significant at Pdose≤0.05. If convergence problems arose due to sparseness of data, Fisher's exact test (binomial data; Proc FREQ in SAS) or Wilcoxon's rank-sum test (categorical data; Proc NARIWAY in SAS) was used to evaluate differences between the CON group and the non-CON dosage groups. In all cases except for health observations, differences were deemed significant at Pₜᵣₜ≤0.05 for these primary discrete variables. Additionally, non-CON dosage groups were compared to one another using a significance level of P_{dose}≤0.05. A less stringent Pₜᵣₜ≤0.10 level of significance was used for health observations.

Continuous variables were analyzed using a linear mixed model, Proc MIXED SAS version 9.4. Differences were deemed significant at Pₜᵣₜ≤0.05 for the discrete variables. As required by protocol, d −59 BW was included as a covariate in the analysis of the primary non-claim variables (ANCOVA): final BW, HCW, and DMI. This covariate remained in the model regardless of its statistical significance. Contrasts were constructed between the CON group and each non-CON group, and differences were deemed significant at P_{dose}≤0.05. The variables with a statistically significant contrast were tested to determine if dose response followed a linear or quadratic fit. Additionally, non-CON groups were compared to one another using a significance level of P_{dose}≤0.05.

### Results

### Feed Composition and LUB Assays

The ingredient composition, analyzed nutrient content and the formulated nutrient and monensin/tylosin composition of the finishing diet fed during the 56 d treatment phase are presented in Table 5. The LUB assay results (mean of the 11 weekly samples collected during the treatment phase and range) for the CON, 1.5, 3.5, or 5.5 mg·kg⁻¹ treatment groups were 1.34 (1.19 to 1.47), 3.13 (2.89 to 3.56), and 4.93 (4.63 to 5.28) mg·kg⁻¹, respectively, on a 100% DM basis. All LUB feed assay results were within the specified acceptable range. Daily LUB consumption was 0, 13.8, 32.6, and 50.8 mg·hd⁻¹ for the CON, 1.5, 3.5, or 5.5 mg kg⁻¹ treatment groups, respectively.

### Animal Health

Table 6 summarizes the abnormal health observations and animal removals that occurred after treatment initiation. No significant (Pₜᵣₜ>0.10) between-treatment differences were observed. Total removals (animals found dead and all animals removed from the treatment phase for health reasons) for each treatment group were 1.7, 2.5, 1.5, and 1.8% for CON, 1.5, 3.5, or 5.5 mg·kg⁻¹ treatment groups, respectively. All cattle passed routine USDA antemortem inspections. Two steers were not harvested due to hyperactive disposition. One steer (3.5 mg·kg⁻¹ treatment group) was euthanized at the packing plant and not harvested. The second steer (CON group) was returned to the research facility and slaughtered at a later date. Carcass data from these two steers were not included in the analysis.

TABLE 6
Abnormal health observations after treatment initiation
presented as total number and rate (%)

| | | Lubabegron [symbol] dose, mg [symbol] kg [symbol] DM | | | | |
|---|---|---|---|---|---|---|
| Clinical system | Clinical sign | Control | 1.5 | 3.5 | 5.5 | P [symbol] |
| Musculoskeletal | Lameness | 7 (1.0) | 9 (1.3) | 3 (0.4) | 7 (1.0) | 0.444 |
| Gastrointestinal | Bloat [symbol] | 0 | 1 (0.1) | 4 (0.6) | 2 (0.3) | [symbol] |
| Respiratory | Pneumonia | 3 (0.4) | 4 (0. [symbol] ) | [symbol] (0.8) | 2 (0.3) | [symbol] |
| Removals [symbol] | | 12 (1.7) | 18 (2.5) | 11 (1.5) | 13 (1.8) | 0.547 |
| General | Dead [symbol] | 1 (0.1) | 2 (0.3) | 5 (0.7) | 2 (0.3) | 0.379 |

[symbol]

60 hd per pen: 12 pens per treatment; 720 total hd per treatment. Rate (%) of abnormal health was calculated based on 720 hd per treatment. [symbol]
Experior, Blanco, Greenfield, IN, USA. [symbol]
Significant effect: P [symbol] ≤ 0.10. [symbol]
Health observations did not converge, therefore, Fisher's Exact Test for each contrast was performed where animal was the experimental unit.
The total number of animals per dose group was 720 hd. [symbol]
Removals include animals found dead (i.e., System [symbol] General; Sign [symbol] Dead) and all animals removed from study during the treatment phase. [symbol]
Animals found dead. [symbol]
indicates data missing or illegible when filed

### Animal Mobility

Table 7 summarizes the animal mobility assessments that were conducted prior to harvest. No differences (Pₜᵣₜ≥0.17) among treatments for the percent of cattle scored 1 were observed at any time. Cattle scoring a 1 or 2 equaled or exceeded 92% of the animals at all times. Animals receiving abnormal scores of >2 were not different across treatments at any of the assessment time points prior to harvest.

TABLE 7
Animal mobility scores prior to and at harvest in steers
fed lubabgron for the final 56 d of the feeding period

| | | Lubabgron dose, mg · kg⁻¹ DM | | | | |
|---|---|---|---|---|---|---|
| Scoring | Mobility | Percentage of steers | | | | |
| location [symbol] | score [symbol] | Control | 1.5 | 3.5 | 5.5 | P [symbol] |
| Pen | 1 | 86.3 | 85.0 | 85.9 | 86.9 | 0.793 |
| | 2 | 9.9 | 11.4 | 10.4 | 8.3 | 0.299 |
| | >2 | 3.8 | 3.6 | 3.7 | 4.8 | 0.619 |
| Loadout | 1 | 74.6 | 76.6 | 73.7 | 71.3 | 0.170 |
| | 2 | 23.9 | 21.4 | 23.1 | 25.0 | 0.440 |
| | >2 | 1.5 | 2.0 | 3.2 | 3.7 | 0.062 |
| Lairage | 1 | 82.7 | 80.2 | 81.7 | 80.9 | 0.644 |
| | 2 | 10.9 | 11.8 | 12.1 | 12.6 | 0.786 |
| | >2 | 6.4 | 8.0 | 6.2 | 6.5 | 0.523 |

¹Experior, Elanco, Greenfield, IN, USA.
²Animal mobility assessments were conducted at, Approximately 1 wk prior to harvest while cattle were in their home pens (“Pen”), after collection of final pen weights and immediately prior to loading into trucks for slaughter (“Loadout”) and during antemortem inspection during lairage at the packing plant (“Lairage”).
³Cattle mobility was scored by trained observer using a 4-pointt scale from the North American Meat Institute (NAMI, 2015). Score 1 = Normal, walks easily, no apparent lameness, no change in gait. Score 2 = Exhibits minor stiffness, shortness of stride, slight limp, keeps up with normal cattle. Score 3 = Exhibits obvious stiffness, difficulty taking steps, obvious limp, obvious discomfort, lags behind normal cattle. Score 4 = Extremely reluctant to move even when encouraged by a handler, statue-like.
⁴Significant effect: P [symbol] ≤ 0.05. [symbol]
indicates data missing or illegible when filed

### Growth Performance and Carcass Characteristics

Initial BW was not different (Pₜᵣₜ=0.709) for cattle assigned to different treatments. However, final BW and HCW increased (Pₜᵣₜ<0.001) 11.6 to 15.7 kg and 11.3 to 17.1 kg, respectively, in cattle receiving LUB compared to CON (Table 8). Average daily gain increased (Pₜᵣₜ<0.001) 11.3% to 15.5% and DMI increased (Pₜᵣₜ=0.025) 2.0% to 3.0% in

LUB-fed steers compared to CON. Gain efficiency (ADG:DMI) improved (Pₜᵣₜ<0.001) 9.0% to 12.0% for cattle treated with LUB compared to CON. Dressing percentage increased 0.7 to 1.2 percentage points and ribeye area increased 3.3 to 5.7 cm² for LUB-treated cattle compared with those in the CON group (Pₜᵣₜ<0.001). Marbling score decreased 13 to 27 units (Pₜᵣₜ<0.001) and yield grade decreased 7 to 20 points (Pₜᵣₜ<0.001) compared to CON. The frequency of excessively trimmed carcasses was 1.1% (8/707), 1.4% (10/702), 1.8% (13/708), and 1.4% (10/707) for the CON, 1.5, 3.5, or 5.5 mg kg⁻¹ LUB groups, respectively (data not shown). The frequency of dark cutter carcasses was 0.3% (2/707), 0.6% (4/702), 0.3% (2/708), and 0.1% (1/707) for the CON, 1.5, 3.5, or 5.5 mg kg⁻¹ LUB groups, respectively (data not shown). Yield grade 2 increased (Pₜᵣₜ≤0.05) in the 3.5 and 5.5 mg kg⁻¹ LUB groups and yield grade 4 decreased (Pₜᵣₜ≤0.05) for all LUB groups compared to CON (Table 9). The proportion of carcasses grading Select increased (Pₜᵣₜ≤0.05) in the 3.5 and 5.5 mg kg⁻¹ LUB groups and the proportion of carcasses grading Upper 2/3 Choice decreased (Pₜᵣₜ≤0.05) for all LUB groups compared to CON.

TABLE 8
Least squares means for the effects of LUB on growth performance traits
and carcass characteristics of beef cattle over a 56 d treatment period.

| | LUB (mg · kg [symbol] DM) | | | | | | |
|---|---|---|---|---|---|---|---|
| Variable | CON | 1.5 | 3.5 | 5.5 | SEM | P [symbol] | P [symbol] |

Growth performance

| | | | | | | | |
|---|---|---|---|---|---|---|---|
| Initial body weight | 567.7 | 569.0 | 569.5 | 567.6 | 2.3 | 0.709 | Not |
| (BW) [symbol] kg | | | | | | | Determined |
| Final BW [symbol] kg | 667.4 | 679.0 | 683.1 | 681.7 | 2.7 | <0.001 | <0.001 |
| Average daily dry matter | 10.1 | 10.3 | 10.4 | 10.3 | 0.25 | 0.025 | 0.013 |
| [symbol] (DMI) [symbol] | | | | | | | |
| Average daily gain | 1.68 | 1.87 | 1.94 | 1.92 | 0.05 | <0.001 | <0.001 |
| (ADG) [symbol] | | | | | | | |
| Gain:feed, ADG:DMI [symbol] | 0.167 | 0.182 | 0.187 | 0.186 | 0.003 | <0.001 | <0.001 |

Carcass characteristics

| | | | | | | | |
|---|---|---|---|---|---|---|---|
| [symbol] carcass weight | 408.0 | 418.3 | 423.9 | 425.1 | 1.4 | <0.001 | <0.001 |
| (HCW) [symbol] , kg | | | | | | | |
| Dressing percentage [symbol] | 61.1 | 61.8 | 62.1 | 62.3 | 0.11 | <0.001 | <0.001 |
| Adjusted fat thickness, cm | 1.54 | 1.53 | 1.48 | 1.48 | 0.06 | 0.038 | <0.007 |
| Ribeye area, cm² | 93.7 | 97.0 | 99.1 | 99.4 | 1.7 | <0.001 | <0.001 |
| Marbling score [symbol] | [symbol] | 460 | 450 | [symbol] | 4.6 | <0.001 | <0.001 |
| Yield grade [symbol] | 3.21 | 3.14 | 3.02 | 3.01 | 0.15 | <0.001 | <0.001 |
| KPH fat, % | 2.11 | 2.12 | 2.12 | 2.14 | 0.03 | 0.774 | Not |
| | | | | | | | Determined |

| | | Difference from control [symbol] | | |
|---|---|---|---|---|
| | | 1.5 mg · kg [symbol] | 3.5 mg · kg [symbol] | 5.5 mg · kg [symbol] |
| Variable | P [symbol] | vs. control | vs. control | vs. control |
| Growth performance | | | | |
| Initial body weight | Not | 1.3 | 1.8 | −0.1 |
| (BW) [symbol] kg | Determined | | | |
| Final BW [symbol] kg | <0.001 | 11.6 (1.7) [symbol] | 15.7 (2.4) [symbol] | 14.3 (2.1) [symbol] |
| Average daily dry matter | 0.060 | 0.2 (2.0) [symbol] | 0.3 (3.0) [symbol] | 0.2 (2.0) [symbol] |
| [symbol] (DMI) [symbol] | | | | |
| Average daily gain | <0.001 | 0.19 (11.3) [symbol] | 0.26 (15.5) [symbol] | 0.24 (14.3) [symbol] |
| (ADG) [symbol] | | | | |
| Gain:feed, ADG:DMI [symbol] | <0.001 | 0.015 (9.0) [symbol] | 0.028 (12.0) [symbol] | 0.019 (11.4) [symbol] |
| Carcass characteristics | | | | |
| [symbol] carcass weight | <0.001 | 11.3 (2.8) [symbol] | 15.9 (3.9) [symbol] | 17.1 (4.2) [symbol] |
| (HCWT, kg | | | | |
| Dressing percentage [symbol] | 0.014 | 0.7 [symbol] | 1.0 [symbol] | 1.2 [symbol] |
| Adjusted fat thickness, cm | 0.761 | −0.01 [symbol] | −0.08 [symbol] | −0.06 [symbol] |
| Ribeye area, cm² | <0.001 | 3.3 (3.5) [symbol] | 5.4 (5.8) [symbol] | 5.7 (6.1) [symbol] |
| Marbling score [symbol] | 0.097 | −13.0 (−2.8) [symbol] | −23.0 (−4.9) [symbol] | −27.0 (−5.7) [symbol] |
| Yield grade [symbol] | 0.193 | −0.07 [symbol] | −0.19 [symbol] | −0.20 [symbol] |
| KPH fat, % | Not | 0.01 | 0.01 | 0.03 |
| | Determined | | | |

[symbol]

Experior, Elanco, Greenfield, IN, USA. [symbol]
Control values subtracted from treatment values. Values in parenthesis are percent change from Control. [symbol]
Different from Control P ≤ 0.05. [symbol]
Significance for linear contrast. [symbol]
Significance for quadratic contrast. [symbol]
Growth performance and dressing percentage were based on unshrunk initial and final BW. [symbol]
Initial animal weight used as a covariate. [symbol]
400 = Small [symbol] (low choice); 500 = Modest [symbol] (middle choice); 600 = Moderate [symbol] (high choice). Determined by objective measurement with a camera. [symbol]
Yield Grade = 2.50 + (0.98 × adj. fat thickness, cm) + (0.2 × KPH fat, %) + (0.0038 × HCW, kg) [symbol] (0.32 × ribeye area, [symbol] Least square means with different superscripts differ P ≤ 0.05. [symbol]
indicates data missing or illegible when filed

TABLE 9
Distribution of yield grades.

| | CON | 1.5 mg · kg⁻¹ DM LUB | 3.5 mg · kg⁻¹ DM LUB | 5.5 mg · kg⁻¹ DM LUB |
|---|---|---|---|---|
| YG 5 (%) | 2.1 | 2.7 | 1.7 | 2.0 |
| YG 4 (%) | 21.8^{x} | 17.1^{y} | 17.3^{y} | 16.0^{y} |
| YG 3 (%) | 47.3 | 49.0 | 44.0 | 44.2 |
| YG 2 (%) | 24.6^{x} | 25.8^{x} | 31.5^{y} | 31.6^{y} |
| YG 1 (%) | 4.1 | 5.4 | 5.5 | 6.2 |

Discrete yield grade (YG) expressed as a proportion of the cattle in a particular yield grade category to the number cattle graded within each treatment. Within a yield grade category, means from a dose with a different letter differed (P_{dose} ≤ 0.05) from each other. Values represented in this figure are arithmetic means, whereas the denoted differences are between the least squares means calculated using PROC GLIMMIX and represent the probability of cattle in a pen displaying a given response.

### CONCLUSION

Daily dry matter intake was 2.3% greater (Pₜᵣₜ<0.05) for steers that received LUB. Average daily gain was 13.7% greater (Pₜᵣₜ<0.05; 1.68 vs. 1.91 kg), while gain efficiency was 10.8% greater (Pₜᵣₜ<0.05; 0.167 vs. 0.185) for steers fed LUB. Animal mobility was scored in the pen approximately 1 week prior to harvest, when cattle were loaded on trucks scheduled for harvest, and at antemortem inspection during lairage. No treatment differences (Pₜᵣₜ≥0.170) were observed at any time for the percent of cattle receiving mobility scores of 1 or 2 (normal or minor stiffness but moving with the normal cattle, respectively). Cattle mobility scored as a 1 or 2 equaled or exceeded 92% at all times. Final BW and HCW increased (Pₜᵣₜ<0.05) 11.6 to 15.7 kg and 11.3 to 17.1 kg, respectively, in cattle receiving LUB compared to cattle receiving monensin plus tylosin alone.

### Example 3: Effect of Lubabegron Removal Periods on Live Performance and Carcass Characteristics in Beef Steers

This experiment was conducted to evaluate the effect of removing lubabegron from feed for increasing intervals prior to slaughter on growth performance of steers.

### Cattle and Treatments

Seven hundred English x Continental cross steers (initial BW=531±8.8 kg) were used in a randomized complete block design with seven treatments. Pen served as the experimental unit. Treatments included a non-treated control diet that did not contain lubabegron or a lubabegron-containing diet of 3.5 mg/kg DM fed for 56 d with an added voluntary removal of 0, 2, 4, 6, 8, or 16 d prior to harvest (Table 2). Animals were managed as two-time replications with each replication containing five blocks (7 pens/block and 10 steers/pen). All ten blocks started and ended within 48 hours of each other. Cattle were weighed by pen using a 45,000-kg capacity pen scale (Model WI-130, Avery Weigh-Tronix LLC, Fairmont, MN; 3.66 m×21.34 m). All treatments were on feed for 71 d with harvest scheduled for 72 d. Because of an impending weather event, cattle were weighed to obtain a final BW one d early. Cattle were placed on their respective ration for the additional d and then shipped to the harvest facility the following morning.

### Feed and Mixing Procedures

In both studies, diets (Table 3) were formulated to meet or exceed nutrient requirements of finishing beef cattle (NRC, 2016). Mixing procedures and feeding methodologies were similar for both studies. Mixing procedures to ensure homogeneity of feed were confirmed before the start of the experiments and mixer scales were validated before use each day. A stationary mixer (Kirby model 705; 20.0 m³ capacity) was used to prepare diets. Lubabegron was added to the ration via a water flush through a micro-ingredient machine. Feed was transferred into trailer-mounted mixers (Kirby 475; 13.5 m³ capacity) that fed cattle once daily.

### Harvest and Tissue Collection

657 head were transported to a harvest facility approximately 495 km from the research facility. Animals were scored for animal mobility using the North American Meat Institute (NAMI, 2015) scoring scale described by Edwards-Callaway et al., (2017) while in lairage. The mobility scoring system is a 4-point scoring system where 1=normal, walks easily with no apparent lameness or change in gait; 2=keeps up with normal cattle when the group is walking, exhibits 1 or more of the following: stiffness, shortened stride, or slight limp; 3=lags behind normal cattle when the group is walking, exhibits 1 or more of the following: obvious stiffness, difficulty taking steps, obvious limp, or discomfort; 4=extremely reluctant to move, even when encouraged by handlers. Experienced staff collected an ear tag number, plant identification (ID), and carcass sequence and applied a unique carcass ID. Data collected on the kill floor included HCW, instances of excessive trim (>approximately 9 kg), and prevalence of liver abscess (Brown et al., 1975). Other carcass measures were obtained via instrument grading.

The remaining 35 head were shipped to a different facility located approximately 80 km from the research location to facilitate collection of tissues at harvest (liver, muscle, rumen, reticulum, abomasum).

### Feed

Dry matter intake (DMI) was determined by the difference between feed delivered and remaining feed. Diet DM was determined weekly by oven drying (>12 h at 100° C.). Diet samples were collected once per week from a randomly chosen batch of each diet for both nutrient and lubabegron analysis. A composite sample (approximately 2 kg) for each analysis×diet was generated by combining three subsamples collected from each 1/3 of a chosen batch. Composite samples were stored at −20° C. until submission. Composite feed samples were submitted for crude protein (AOAC, 2019; method 990.03), calcium, and phosphorus (AOAC, 2019; methods 968.08 and 985.01) to MVTL Laboratories (New Ulm, MN).

### Statistical Analysis

Continuous variables were analyzed using the Mixed procedure of SAS (version 9.4; SAS Institute, Cary, NC). Block served as a random effect whereas treatment was fixed. Initial body weight was used to describe the uniformity of cattle randomized to treatment and used as a covariate when appropriate. Data were calculated with deads and removals excluded. Linear and quadratic effects of lubabegron removal were conducted as were individual comparisons for duration of removal (i.e., 0 vs 2 d; 0 vs 4 d, 0 vs 6 d; 0 vs 8 d and 0 vs 16 d). Fisher's Exact test was evaluated on mobility scores 1 and 2. No test was conducted on mobility scores of 3 or 4 because no scores were reported. Simple means and standard deviation of the tissues collected for the 5 animals were calculated.

### Results

Cattle within the 0-d treatment had a withdrawal of either 1 d 0 h 4 m or 1 d 1 h 49 m due to transit time to facility and time in the lairage. The smaller group of 35 animals had a 17 h 21 m removal duration.

The results of withdrawal periods on growth performance are shown in Table 10 Carcass weight was lower (395 kg) for the non-treated cattle and was less (P<0.001) compared to similarly treated cattle within the 0 d removal group (409 kg). Carcass weight for cattle receiving lubabegron ranged from 408 to 413 kg among the other treatments. Dressing percentage followed a similar pattern as HCW. Dressing percent was 61.7% for cattle in the non-treated group and ranged from 62.6% to 63.0% in the other treatment groups. Marbling score was reduced (P<0.001) for cattle fed lubabegron. Marbling score (551) was higher (P<0.001) in the non-treated group compared to a range of marbling scores of 482 to 491 in the medicated groups. Ribeye area (REA) increased (P<0.001) in cattle fed lubabegron cattle. Carcass REA averaged 83.1 cm² for the non-treated group while REA ranged from 89.7 to 91.6 cm² for the other treatments. Calculated yield grade data was leaner (P<0.01) with lubabegron. Calculated yield grade was improved (P<0.001; 3.44 vs 3.08) by 10.5% comparing the non-treated group to cattle fed lubabegron for 56 d with a 0 d removal. Calculated yield grade ranged from 3.06 to 3.21 for all lubabegron treated groups. Again, there was no effect (P>0.05) of removal duration on any measured carcass parameter.

TABLE 10
Effect of lubabegron and removal duration on carcass measures.

| | Lubabegron mg/kg: | | | | | | | | | | | | | |
|---|---|---|---|---|---|---|---|---|---|---|---|---|---|---|
| | 0 | 3.5 | 3.5 | 3.5 | 3.5 | 3.5 | 3.5 | | | | | | | |
| | Removal duration: | | | | | | | Contrast¹ | | | | | | |
| | NA | 0 | 2 | 4 | 6 | 8 | 16 | SEM² | P_{TRT} | P_{LUBA} | P_{LIN} | P_{QUAD} | P_{0 VS 8} | P_{0 VS 16} |
| HCW³⁴, kg | 395 | 409 | 411 | 413 | 410 | 413 | 498 | 1.7 | <0.001 | <0.001 | 0.39 | 0.04 | 0.12 | 0.63 |
| Dressing⁴⁵% | 61.7 | 62.7 | 62.7 | 62.8 | 62.9 | 63.0 | 62.6 | 0.18 | <0.001 | <0.001 | 0.74 | 0.16 | 0.26 | 0.65 |
| Marbling⁴⁶ | 551 | 482 | 491 | 488 | 483 | 483 | 484 | 19.4 | <0.001 | <0.001 | 0.79 | 0.99 | 0.90 | 0.80 |
| Ribeye Area cm² | 81.1 | 91.0 | 90.3 | 90.3 | 91.6 | 90.3 | 89.0 | 0.87 | <0.001 | <0.001 | 0.64 | 0.86 | 0.59 | 0.24 |
| Calculated YG | 3.44 | 3.08 | 3.20 | 3.21 | 3.06 | 3.14 | 3.18 | 0.086 | <0.001 | <0.001 | 0.63 | 0.86 | 0.49 | 0.24 |

¹PTRT—Treatment differences; PLUBA—Lubabegron treatment difference (TC_A vs TC_B); PLIN—Linear trend within removal duration; PQUAD—Quadratic trend with removal duration; P0 vs 8—comparison of 0-vs 8-d removal treatments; P0 vs 16 comparison of 0-vs. 16-d removal treatments
²SEM: Standard Error of Treatment Mean
³Hot Carcass Weight
⁴Initial weight used as a covariate
⁵Dressing Percent: (Hot Carcass Weight/Final Liveweight with 4% shrink) * 100
⁶Marbling Score: 500 = Small

Animal mobility was assessed on cattle presented for harvest (Table 11). Across all treatments approximately 97.3% scored a 1 while 2.7% scored a 2 during evaluation. No differences (P<0.25) were noted when lubabegron was fed although mobility tended (P<0.06) to be reduced with increased duration of removal. Four animals died during the experiment. Three died from bloat while one died from a posterior vena cava thrombosis. Four animals were removed due to respiratory disease (2), lameness (1), and an incorrect pen assignment (1). These losses did not appear to be treatment associated.

The current experiment demonstrates the effects on BW and HCW are not affected by a duration of lubabegron removal up to 16 d. This was surprising because the effect of duration of removal on animal performance of beta-adrenergic receptor blockers is mixed. Bryant et al. (2010) did not report a reduction in BW or HCW with increasing removal duration whereas Rincker et al. (2021) demonstrated a −0.4 kg/d reduction in HCW with increased duration of removal. In both studies, the removal duration was up to 8 d. Holland et al. (2010) fed zilpaterol at 8.3 mg/kg diet DM and followed by removal duration of 3, 10, 17, or 24 d prior to harvest, Final and carcass-adjusted final BW were not affected (P≥0.14) by feeding zilpaterol. They noted a difference in HCW between zilpaterol fed and non-treated cattle of 14, 17, 5, and 6 kg with 3, 10, 17, and 24 d removal, respectively. With extended periods of removal, treated animals tended to mimic those of the non-treated cattle.

TABLE 11
Effect of lubabegron and removal duration on animal mobility at harvest. Study 2¹

| | Lubabegron, mg/kg | | | | | | | | | | | | |
|---|---|---|---|---|---|---|---|---|---|---|---|---|---|
| | 0 | 3.5 | 3.5 | 3.5 | 3.5 | 3.5 | 3.5 | | | | | | |
| | Removal Duration, d | | | | | | | Contrasts² | | | | | |
| Mobility Score | NA | 0 | 2 | 4 | 6 | 8 | 16 | P_{TRT} | P_{LUBA} | P_{LIN} | P_{QUAD} | P_{0 VS 8} | P_{0 VS 16} |

| | | | | | | | | | | |
|---|---|---|---|---|---|---|---|---|---|---|
| 1, n | 92 | 92 | 95 | 91 | 89 | 87 | 90 | 0.25 | 0.72 | 0.72 |
| 2, n | 0 | 3 | 0 | 2 | 4 | 4 | 5 | 0.25 | 0.72 | 0.72 |
| 3³, n | 0 | 0 | 0 | 0 | 0 | 0 | 0 | | | |
| 4³, n | 0 | 0 | 0 | 0 | 0 | 0 | 0 | | | |

¹North American Meat Institute. Mobility Scoring System: 1 = Normal, walks easily with no apparent lameness or change in gait; 2 = Keeps up with normal cattle when the group is walking, exhibits 1 or more of the following: stiffness, shortened stride, or slight limp; 3 = Lags behind normal cattle when the group is walking, exhibits 1 or more of the following: obvious stiffness, difficulty taking steps, obvious limp or discomfort; 4 = Extremely reluctant to move, even when encouraged by handlers.
²P_{TRT}—Treatment differences; P_{LUBA}—Lubabegron treatment difference; P_{LIN}—Linear trend within removal duration; P_{QUAD}—Quadratic trend with removal duration; P_{0 vs 8}—comparison of 0- vs 8-d removal treatments; P_{0 vs 16} comparison of 0- vs. 16-d removal treatments.
³Fisher's Exact Test was not performed where groups had identical mobility scores reported

### REFERENCE LIST

Each article is herein incorporated by reference in its entirety.
Brown, H., R. F. Bing, H. P. Grueter, J. W. McAskill, C. O. Cooley and R. P. Rathmacher. 1975. Chlorotetracycline for the prevention of liver abscesses, improved weight gain and feed efficiency in feedlot cattle. J. Anim. Sci. 40:207. doi.org/10.2527/jas 1975.402207x.
Brown, M. S., N. A. Cole, S. Gruber, J. Kube, and J. S. Teeter. 2019. Modeling and prediction accuracy of ammonia gas emissions from feedlot cattle. Applied Anim. Sci. 35:347. doi/10.15232/aas.2018-01934.
Edwards-Callaway, L. N., M. S. Calvo-Lorenzo, J. A. Scanga, and T. Grandin. 2017. Mobility scoring of finished cattle. Vet. Clin. North Am. Food Anim. Pract. 33:235-250. doi:10.1016/j.cvfa.2017.02.006
Guelker, M. R., A. N. Haneklaus, J. C. Brooks, C. C. Carr, R. J. Delmore Jr, D. B. Griffin, D. S. Hale, K. B. Harris, G. G. Mafi, D. D. Johnson, et al. 2013. National Beef Tenderness Survey-2012: Warner-Bratzler shear force values and sensory panel ratings for beef steaks from United States retail and food service establishments. J. Anim. Sci. 91:1005-1014. doi:10.2527/jas2012-5785
Holland, B. P., C. R. Krehbiel, G. G. Hilton, M. N. Streeter, D. L. VanOverbeke, J. N. Shook, D. L. Step, L. O. Burciaga-Robles, D. R. Stein, D. A. Yates, J. P. Hutcheson, W. T. Nichols, and J. L. Montgomery. 2010. Effect of extended withdrawal of zilpaterol hydrochloride on performance and carcass traits in finishing beef steers. J. Anim. Sci.88:338. doi:10.2527/jas.2009-1798
Howard, S. T., D. R. Woerner, J. A. Scanga, D. L. VanOverbeke, G. G. Mafi, J. L. Igo, M. D. Salman, J. D. Tatum, and K. E. Belk. 2013. North American Beef Tenderness Survey 2011-2012: benchmarking tenderness and sample shipping procedures. J. Anim. Sci. 91:5981-5988. doi:10.2527/jas.2013-7040
Igo, M. W., A. N. Arnold, R. K. Miller, K. B. Gehring, L. N. Mehall, C. L. Lorenzen, R. J. Delmore Jr, D. R. Woerner, B. E. Wasser, and J. W. Savell. 2015. Tenderness assessment of top loin steaks from retail markets in four U. S. cities. J. Anim. Sci. 93:4610-4616. doi:10.2527/jas.2015-9085
Martinez, H. A., A. N. Arnold, J. C. Brooks, C. C. Carr, K. B. Gehring, D. B. Griffin, D. S. Hale, G. G. Mafi, D. D. Johnson, C. L. Lorenzen, et al. 2017. National beef tenderness survey-2015: palatability and shear force assessments of retail and foodservice beef. Meat Muscle Biol. 1. doi:10.22175/mmb2017.05.0028
NAMI. 2015. Mobility scoring for cattle. North American Meat Institute [accessed Jun. 10, 2020]. https://www.youtube.com/watch?v=QIslfHCvkpg.
NASEM. 2016. National academies of science engineering and medicine. Nutrient requirements of beef cattle. 8th Rev. Ed. Washington (DC): National Academies Press
Platter, W. J., J. D. Tatum, K. E. Belk, P. L. Chapman, J. A. Scanga, and G. C. Smith. 2003. Relationships of consumer sensory ratings, marbling score, and shear force value to consumer acceptance of beef strip loin steaks. J. Anim. Sci. 81:2741-2750. doi:10.2527/2003.81112741x
Rincker, P. J., J. B. Allen, M. Edmonds, M. S. Brown, and J. C. Kube, 2021. Effects of voluntary removal of ractopamine hydrochloride (Optaflexx) on live performance and carcass characteristics of beef steers, Tran. Anim. Sci. 5:2 doi:10.1093/tas/txab047
USDA. 1997. United States standards for grades of carcass beef. Washington (DC): Agric. Marketing Service, USDA
Vasconcelos, J. T., N. A. Cole, K. W. McBride, A. Gueye, M. L. Galyean, C. R. Richardson, and L. W. Greene. 2009. Effects of dietary crude protein and supplemental urea levels on nitrogen and phosphorus utilization by feedlot cattle. J. Anim. Sci. 87:1174-1183. doi:10.2527/jas.2008-1411

1. A method for improving growth performance of feedlot cattle comprising administering to the cattle an effective amount of lubabegron or a physiologically acceptable salt thereof at a dose of between 3.5 and 22 mg*kg⁻¹ dry matter.

2. The method of claim 1, wherein the lubabegron or a physiologically acceptable salt thereof is administered at a dose of 5.5 mg*kg⁻¹ dry matter.

3. The method of claim 1, wherein the administration is for 56 to 91 days.

4. The method of claim 3, wherein the administration is for the last 56 to 91 days of the finishing period.

5. The method of claim 1, wherein the lubabegron is removed for a period of from between 2 and 16 days prior to harvest, and wherein the improved growth performance is not significantly attenuated by the removal of lubabegron.

6. The method of claim 1, wherein the lubabegron is removed for a period of from between 2 and 16 days prior to harvest, and wherein final body weight (BW), hot carcass weight (HCW), average daily gain (ADG), gain to feed ratio (G:F), dry matter intake (DMI), dressing percentage, ribeye area or longissimus dorsi muscle area (LM area), yield grade (YG), and/or yield grade distribution are not significantly attenuated by the removal of lubabegron.

7. The method of claim 5, wherein the lubabegron is removed 16 days prior to harvest.

8. The method of claim 1, wherein the administration increases final body weight (BW) by at least 11 kg when compared to control cattle not administered lubabegron.

9. The method of claim 1, wherein the administration results in an increase of hot carcass weight (HCW) by at least 15 kg when compared to control cattle not administered lubabegron.

10. The method of claim 1, wherein the administration results in an increase in average daily gain (ADG) of between about 11% and 16% when compared to control cattle not administered lubabegron.

11. The method of claim 1, wherein the administration increases the gain to feed (G:F) ratio by at least 10% when compared to control cattle not administered lubabegron.

12. The method of claim 1, wherein the administration increases dry matter intake (DMI) by the cattle by about 2% to 3% when compared to control cattle not administered lubabegron.

13. The method of claim 1, wherein the administration increases the ribeye area when compared to control cattle not administered lubabegron.

14. The method of claim 1, wherein the administration increases the dressing percentage when compared to control cattle not administered lubabegron.

15. The method of claim 14, wherein the dressing percentage is increased by about 0.9 to 1.3 units.

16. The method of claim 1, wherein the administration decreases yield grade (YG) when compared to control cattle not administered lubabegron.

17. The method of claim 16, wherein the YG decreases by about 10% to 11% when compared to control cattle not administered lubabegron.

18. The method of claim 1, wherein the administration improves yield grade distribution when compared to control cattle not administered lubabegron.

19. The method of claim 18, wherein a greater percentage of cattle are Yield Grade 2 when compared to control cattle not administered lubabegron.

20. The method of claim 18, wherein a lower percentage of cattle are Yield Grade 4 when compared to control cattle not administered lubabegron.

21. The method of claim 1, wherein the wherein the physiologically acceptable salt is lubabegron fumarate.

22. The method of claim 1, wherein the lubabegron or the physiologically acceptable salt thereof is administered in feed.

23. A method of reducing kidney pelvic and heart fat (KPH) in feedlot cattle comprising administering to the cattle an effective amount of lubabegron or a physiologically acceptable salt thereof.

24. The method of claim 23, wherein the lubabegron or a physiologically acceptable salt thereof is administered in a dose of 22 mg*kg⁻¹.

25. The method of claim 23, wherein the physiologically acceptable salt is lubabegron fumarate.
